(19) United States
(12) Patent Application Publication
(10) Pub. No.: US 2006/0078506 A1
(43) Pub. Date: Apr. 13, 2006

(54) Methods, systems and devices for noninvasive pulmonary delivery

(76) Inventors: Ralph Niven, Half Moon Bay, CA (US); Wiwik S. Watanabe, Mountain View, CA (US); Matthew K. Thomas, Cambridge, MA (US); David Brown, St. Petersburg, FL (US); Mark Johnson, Los Alto, CA (US); Maithili Rairkar, San Jose, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/130,783

(22) Filed: May 17, 2005

Related U.S. Application Data
(60) Provisional application No. 60/573,570, filed on May 20, 2004.
(60) Provisional application No. 60/639,503, filed on Dec. 27, 2004.
(60) Provisional application No. 60/673,155, filed on Apr. 20, 2005.

(51) Int. Cl. A61L 9/04; A61K 9/14; A61K 38/17; A61M 11/00

(52) U.S. Cl. 424/45; 424/46; 128/200.23; 514/2

(57) ABSTRACT

The invention is directed to noninvasive methods, systems and devices for pulmonary delivery of aerosolized active agents and methods of treating respiratory dysfunction.

### CROSS-REFERENCE TO RELATED APPLICATIONS

This application claims priority to U.S. Application No. 60/573,570, filed May 20, 2004, U.S. Application No. 60/639,503 filed Dec. 27, 2004 and U.S. Application No. 60/673,155, filed Apr. 20, 2005, the disclosures of which are incorporated by reference in their entireties.

### FIELD

The invention is directed to noninvasive methods, systems and devices for pulmonary delivery of aerosolized active agents and methods of treating respiratory dysfunction.

### BACKGROUND

Pre- and full-term infants born with a respiratory dysfunction, which includes but is not limited to, respiratory distress syndrome (RDS), meconium aspiration syndrome (MAS), persisten pulmonary hypertension (PHN), acute respiratory distress syndrome (ARDS), PCP, TTN and the like often require prophylactic or rescue respiratory support. Infants born at 28 weeks or less are almost universally intubated and mechanically ventilated. There is a significant risk of failure during the process of intubation and a finite chance of causing damage to the upper trachea, laryngeal folds and surrounding tissue. Mechanical ventilation over a prolonged time, particularly where elevated oxygen tensions are employed, can also lead to acute lung damage. If ventilation and oxygen is required for prolonged periods of time and/or if the ventilator is not sufficiently managed, the clinical consequences can include broncho pulmonary dysplasia, chronic lung disease, pulmonary hemorrhage, intraventricular hemorrhage, and periventricular leukomalacia.

Infants born of larger weight or gestational age who are not overtly at risk of developing respiratory distress may be supported by noninvasive means. One approach is nasal continuous positive airway pressure (nCPAP or CPAP). CPAP is a means to provide voluntary ventilator support while avoiding the invasive procedure of intubation. CPAP provides humidified and slightly over-pressurized gas (approximately 5 cm H₂O above atmospheric pressure) to an infant's nasal passageway utilizing nasal prongs or a tight fitting nasal mask. CPAP also has the potential to provide successful treatment for adults with various disorders including chronic obstructive pulmonary disease (COPD), sleep apnea, ARDS/ALI and the like.

In addition to respiratory support, infants are often treated with exogenous surfactant, which improves gas exchange and has had a dramatic impact on mortality. Typically, the exogenous material is delivered as a liquid bolus to the central airways via a catheter introduced through an endotracheal tube.

There are three problems associated with the current methods of surfactant delivery. First, there is the potential for trauma associated with using an endotracheal tube in conjunction with mechanical ventilation. Second, there is the potential for damage associated with high oxygen and pressure settings. Third, the process of delivering via liquid bolus may cause temporary airway plugging which can lead to a transient reduction in circulatory oxygen saturation and hemodynamic changes. These changes can lead to systemic issues such as intraventricular hemorrhaging. The instilled bolus must be aspirated effectively and simultaneously flow and spread across the lung surfaces.

In addition, after compression of surfaces at the end of expiration, it is essential that the surfactant be capable of respreading over surfaces as the lungs expand during an inspiratory maneuver. When delivered as a liquid bolus, the surfactant often does not have effective respreadability capacity.

With these issues in mind, attempts have been made to administer surfactant in a more “gentle” way, such as by aerosolization. However, thus far attempts to deliver surfactant as an aerosol simultaneously with CPAP have proved unsuccessful due to the lack of sufficient quantities of surfactant reaching the lungs (Berggren et al., Acta Poediatr. 2000, 89:460-464). This is due to inefficient delivery caused by deposition of aerosolized material on sites external to the lungs. A significant contributor to these extrathoracic losses is material deposited at or around the nasal prongs or mask where there may be the potential to clog the prongs during extended delivery periods. It is also a known problem that the rate at which aerosolized surfactant deposits on the lung surface may be low relative to the rate at which it is cleared. Clearance rates are also likely to be accelerated in lungs with ongoing inflammatory disease. Thus, no opportunity exists for exogenous surfactant to accumulate within the lung environment and exert a therapeutic effect. In general, the absolute quantities of surfactant administered and deposited in a practical time frame may also be too small to have a significant therapeutic impact.

The same problems occur when attempting to deliver other high dose therapeutics via pulmonary routes such as antibiotics, protease inhibitors,

In light of the difficulty of delivering surfactant as an aerosol, there is an ongoing need to provide a method for safe, effective aerosol delivery of high dose therapeutics such as surfactant or other active agents.

### SUMMARY OF THE INVENTION

This invention is directed to noninvasive pulmonary delivery of an active agent to a mammalian patient and especially human infant patients in need of respiratory treatment. Methods are provided for delivering an aerosolized active agent to a patient. Preferred embodiments generally begin with the steps of obtaining an active agent as a mixture in a medium, and generating a stream of particles of the mixture with an aerosol generator to produce the aerosolized active agent desired for delivery. In accordance with one preferred method embodiment, the aerosolized active agent is communicated to and through a novel fluid flow connector. The connector is preferably configured to direct the aerosolized active agent along a main aerosol flow path and to an outlet, and to be able to collect deposits in an area that is, preferably, located at least partially outside the main aerosol flow path. One suitable location for collecting deposits within the connector is an area that is spaced apart from the connector outlet.

Deposits that are collected in the fluid flow connector can be retrieved from the connector at various junctures contemplated by the methods of the present invention. For example, a first aerosolized active agent can be delivered to a patient, the deposits retrieved from the fluid flow connector, and then a second aerosolized active can be delivered to the patient. The deposits containing a portion of active agent can be delivered to a patient substantially in its collected form, such as, for example, via a syringe dosed through a patient's nares, or can be re-aerosolized and then delivered to the patient.

In accordance with another preferred method embodiment, the aerosolized active agent is impacted with a stream of gas. The stream of gas is preferably directed toward the aerosolized active agent in a radially symmetric manner. The stream of gas can affect the aerosolized active agent in any number of ways. For example, the impacting stream of gas can alter the characteristics of a first aerosol to produce a second aerosol, which is then delivered to the patient. The mass median aerodynamic diameter of particles associated with the second aerosol may be smaller than that of the particles associated with the first aerosol. The ratio of active agent to medium may be greater in the second aerosol as compared to that in the first aerosol. The stream of gas can affect the aerosolized active agent physically. For example, the impacting stream of gas can direct the aerosol flow path through one or more remaining connectors or conduits before reaching the patient.

Systems for delivering an aerosolized active agent to a patient are also provided. In accordance with one preferred embodiment, a system includes an aerosol generator for forming the aerosolized active agent, a delivery means, and a trap interposed between the aerosol generator and the delivery means for collecting deposits separated from the aerosolized active agent. At least a portion of the trap is preferably positioned substantially outside a main aerosol flow path.

In accordance with another preferred system embodiment, the system includes an aerosol generator, a fluid flow connector connected to the aerosol generator, and optionally, a pair of nasal prongs connected to a delivery outlet of the fluid flow connector. The fluid flow connector includes a chamber, an aerosol inlet, a delivery outlet, and a trap for collecting deposits associated with the aerosolized active agent. An aerosol flow path is defined between the aerosol inlet and the delivery outlet. The aerosol flow path is preferably devoid of angles less than 90°. Each of the pair of nasal prongs has an internal diameter that is preferably less than or equal to about 10 mm.

Fluid flow connectors adapted for delivery of an aerosolized active agent are also provided. The fluid flow connectors are suitable for use in both the above preferred methods and systems, and methods and systems other than those shown and described herein. In accordance with one preferred connector embodiment, the connector includes a chamber having an aerosol inlet, a delivery outlet, an aerosol flow path defined between the inlet and outlet, and an area for collecting deposits associated with the aerosolized active agent. The deposit collection area is preferably located at least partially outside of the aerosol flow path so that deposits can be collected and substantially isolated from the aerosolized active agent flowing through the connector.

In accordance with another preferred connector embodiment, the connector includes a chamber having an aerosol inlet, a delivery outlet, an aerosol flow path defined between the inlet and outlet, and a means for keeping deposits associated with the aerosolized active agent separated from the aerosol flow path. The means can include a concavity defined in the chamber. The means can also include a lip disposed proximate the delivery outlet.

In accordance with yet another preferred connector embodiment, the connector includes a chamber, an aerosol inlet, a delivery outlet, and an aerosol flow path extending from the inlet to the outlet. The aerosolized active agent preferably flows through the flow path at an angle that is less than about 90°.

In accordance with another preferred connector embodiment, the connector includes a chamber having an aerosol inlet, a delivery outlet, and an internal surface on which deposits associated with the aerosolized active agent can impact. The internal surface is configured for either trapping the deposits and/or facilitating the communication of the deposits to the delivery outlet.

An alternative connector embodiment includes a chamber having an aerosol inlet, a delivery outlet, a ventilation gas inlet and a ventilation gas outlet. The aerosol inlet and the delivery outlet are substantially parallel to each other. And the aerosol inlet can be laterally offset from the delivery outlet.

The methods, systems and devices of the present invention provide for the delivery of an aerosolized active agent to a patient. In an exemplary embodiment of the present invention, the aerosolized active agent is aerosolized lung surfactant delivered at a rate of from about 0.1 mg/min of lung surfactant, measured as total phospholipid content (“TPL”), to about 300 mg/min of surfactant TPL.

Using the methods, systems, and devices of the present invention, a high fraction of aerosolized active agent can be delivered to the patient and deposited in the lungs of the patient. In an exemplary embodiment, greater than about 10% of aerosolized lung surfactant TPL that is in the delivery device exits the device and is delivered to the patient. In a particularly preferred embodiment equal to or greater than about 10%, about 15%, about 20% or about 25% of aerosolized lung surfactant TPL that is in the delivery device exits the device and is delivered to the patient. In one aspect of the invention, equal to or greater than about 2 mg/kg (based on the total weight of the patient) of lung surfactant TPL is deposited in the lungs of the patient. In another aspect, from about 2 mg/kg of lung surfactant TPL to about 175 mg/kg of lung surfactant TPL is deposited in the lungs of the patient.

The present invention provides methods of treating respiratory dysfunction. The amount of aerosolized active agent deposited in the lungs of the patient, using these methods, will be effective to treat respiratory dysfunction in the patient. In a particularly preferred embodiment, the present invention provides methods of treating RDS in infants. The amount of aerosolized active agent deposited in the lungs of these patients will be sufficient for the rescue and/or prophylactic treatment of these patients, i.e., there will be no need for surfactant administration using an endotracheal tube.

### BRIEF DESCRIPTION OF THE DRAWINGS

The invention will be described in greater detail with reference to the preferred embodiments illustrated in the accompanying drawings, in which like elements bear like reference numerals, and wherein:

FIG. 1 illustrates in schematic view a representative system which may be used in conjunction with the methods of the present invention.

FIG. 2 illustrates in schematic view an alternative embodiment of the system used in conjunction with the methods of the present invention when the active agent and the medium is a premix.

FIG. 3 illustrates a partial cross-sectional partially-exploded view of the nebulizer and the conditioning vessel.

FIG. 3A illustrates a cross-sectional view of the CPAP adaptor that is coupled with the conditioning vessel when CPAP is administered simultaneously with the aerosolized active agent.

FIG. 4 illustrates a cross-sectional view of the portion of the conditioning gas unit indicated by the section lines 4-4 in FIG. 3.

FIG. 5 illustrates a plan view of the conditioning gas unit.

FIG. 6 illustrates a plan side perspective view of the conditioning gas unit and a plan side perspective view of the conditioning compartment. FIG. 6A illustrates an upward-looking side perspective view of the unit and compartment with the bottom plate of the unit removed. FIG. 6B illustrates the same upward-looking side perspective view of FIG. 6A with the bottom plate in place.

FIG. 7 illustrates a cross-sectional view of the portion of the conditioning gas unit indicated by the section lines 7-7 in FIG. 3.

FIG. 8 illustrates in schematic form the aerosol traveling from the nebulizer and through the conditioning vessel while being bounded, shaped and directed by the conditioning gas.

FIG. 9 illustrates in schematic form a way to effect simultaneous administration of CPAP and delivery of the aerosol, in which the two components are admixed just prior to delivery to patient. FIG. 9A illustrates a cross-sectional view of the nasal prongs utilizing the delivery method described.

FIG. 10 illustrates in schematic form a second way to effect simultaneous administration of CPAP and delivery of the aerosol, in which the aerosol is delivered via one nasal prong and the CPAP is delivered via the other nasal prong. FIG. 10A illustrates a cross-sectional view of the nasal prongs utilizing the delivery method described.

FIG. 11 illustrates in schematic form a third way to effect simultaneous administration of CPAP and delivery of the aerosol, in which the two components are delivered separately yet coaxially into each of the nasal prongs. FIG. 11A illustrates a cross-sectional view of the nasal prongs utilizing the delivery method described.

FIG. 12 illustrates in schematic form an exemplary system of this invention. FIG. 12A illustrates the exemplary system of FIG. 12 in use with an infant.

FIG. 13 illustrates a comparison of collection rates of aerosolized surfactant in an unconditioned system and aerosolized surfactant in a conditioned system.

FIG. 14 illustrates a comparison of collection rates of conditioned aerosol with varying conditioning gas flow rates and temperatures.

FIG. 15 illustrates a comparison of percentages of collection efficiency of conditioned aerosol with varying conditioning gas flow rates and temperatures.

FIG. 16 illustrates changes in conditioned aerosol volume median diameter when the conditioning gas temperature and flow rate is varied.

FIG. 17 illustrates the size distribution of conditioned aerosol when the conditioning gas flow rate and temperature is varied.

FIG. 18 is a perspective view of one preferred fluid flow connector embodiment in accordance with the present invention.

FIG. 19 is a bottom view of the fluid flow connector shown in FIG. 18.

FIG. 20 is a side view of the fluid flow connector shown in FIG. 18.

FIG. 21 is a cross-sectional view of the fluid flow connector taken through line XXI-XXI in FIG. 18.

FIG. 22 is a cross-sectional view of a second preferred fluid flow connector embodiment provided by the present invention.

FIG. 23 is a cross-sectional view of a third preferred fluid flow connector embodiment of the present invention.

FIG. 24 is a perspective view of a fourth preferred fluid flow connector of the present invention. This embodiment includes a aerosol conditioning vessel.

FIG. 25 is a cross-sectional view of the fluid flow connector shown in FIG. 24.

FIG. 26 is a top perspective view of an exemplary aerosol conditioning vessel in accordance with the present invention.

FIG. 27 is a partial cross-sectional view of an exemplary aerosol concentration chamber connected to a fluid flow connector of the present invention.

FIG. 28 is a partial cross-sectional view of an exemplary deposit collection reservoir in accordance with the present invention.

FIG. 29 illustrates a comparison of percentages of surfactant delivered to infants using an exemplary device of the present invention as compared to a T-adapter.

FIG. 30 illustrates amounts of aerosolized lung surfactant delivered with different size nasal prongs.

FIG. 31 illustrates delivery efficiencies of aerosolized active agents, in conjunction with varied ventilator gas flow rates, through preferred connectors of the present invention.

FIG. 32 illustrates amounts of KL4 lung surfactant delivered to a patient's lungs at varied aerosol generator output rates.

### DETAILED DESCRIPTION

The present invention provides, inter alia, methods and systems for pulmonary delivery of one or more active agents to a patient, devices for the delivery of such agents, and methods for treating respiratory dysfunction in a patient.

Unless otherwise indicated the terminology used herein is for the purpose of describing particular embodiments only and is not intended to limit the scope of the present invention. It must be noted that as used herein and in the claims, the singular forms “a,” “and” and “the” include plural referents unless the context clearly dictates otherwise. In this specification and in the claims which follow, reference will be made to a number of terms which shall be defined to have the following meanings:

“Mass median aerodynamic diameter” or “MMAD” of an aerosol refers to the aerodynamic diameter for which half the particulate mass of the aerosol is contributed by particles with an aerodynamic diameter larger than the MMAD and half by particles with an aerodynamic diameter smaller than the MMAD. This can be measured using, for example, inertial cascade impaction techniques or by sedimentation methods.

In accordance with preferred embodiments, the present invention facilitates the delivery of one or more active agents as a mixture in a medium. As used herein the term “mixture” means a solution, suspension, dispersion or emulsion. “Emulsion” refers to a mixture of two or more generally immiscible liquids, and is generally in the form of a colloid. The mixture can be of lipids, for example, which may be homogeneously or heterogeneously dispersed throughout the emulsion. Alternatively, the lipids can be aggregated in the form of, for example, clusters or layers, including monolayers or bilayers. “Suspension” or “dispersion” refers to a mixture, preferably finely divided, of two or more phases (solid, liquid or gas), such as, for example, liquid in liquid, solid in solid, gas in liquid, and the like which preferably can remain stable for extended periods of time. Preferably, the dispersion of this invention is a fluid dispersion.

The mixture comprises the active agent at a desired concentration and a medium. Preferably, the concentration of the active agent in the medium is selected to ensure that the patient is receiving an effective amount of active agent and can be, for example, from about 1 to about 100 or about 120 mg/ml.

Based on the active agent chosen and the medium, one of skill in the art is readily able to determine the proper concentration. Mixtures delivered using the present invention often include one or more wetting agents. The term “wetting agent” means a material that reduces the surface tension of a liquid and therefore increases its adhesion to a solid surface. Preferably, a wetting agent comprises a molecule with a hydrophilic group at one end and a hydrophobic group at the other. The hydrophilic group is believed to prevent beading or collection of material on a surface, such as the nasal prongs. Suitable wetting agents are soaps, alcohols, fatty acids, combinations thereof and the like.

The term “active agent” as used herein refers to a substance or combination of substances that can be used for therapeutic purposes (e.g., a drug), diagnostic purposes or prophylactic purposes via pulmonary delivery. For example, an active agent can be useful for diagnosing the presence or absence of a disease or a condition in a patient and/or for the treatment of a disease or condition in a patient. “Active agent” thus refers to substances or combinations of substances that are capable of exerting a biological effect when delivered by pulmonary routes. The bioactive agents can be neutral, positively or negatively charged. Exemplary agents include, for example, insulins, autocoids, antimicrobials, antipyretics, antiinflammatories, surfactants, antibodies, antifungals, antibacterials, analgesics, anorectics, antiarthritics, antispasmodics, antidepressants, antipsychotics, antiepileptics, antimalarials, antiprotozoals, anti-gout agents, tranquilizers, anxiolytics, narcotic antagonists, antiparkinsonisms, cholinergic agonists, antithyroid agents, antioxidants, antineoplastics, antivirals, appetite suppressants, antiemetics, anticholinergics, antihistaminics, antimigraines, bone modulating agents, bronchodilators and anti-asthma drugs, chelators, antidotes and antagonists, contrast media, corticosteroids, mucolytics, cough suppressants and nasal decongestants, lipid regulating drugs, general anesthetics, local anesthetics, muscle relaxants, nutritional agents, parasympathomimetics, prostaglandins, radio-pharmaceuticals, diuretics, antiarrhythmics, antiemetics, immunomodulators, hematopoietics, anticoagulants and thrombolytics, coronary, cerebral or peripheral vasodilators, hormones, contraceptives, diuretics, antihypertensives, cardiovascular agents such as cardiotonic agents, narcotics, vitamins, vaccines, and the like.

Preferably, the active agent employed is a high-dose therapeutic. Such high dose therapeutics would include antibiotics, such as amikacin, gentamicin, colistin, tobramycin, amphotericin B. Others would include mucolytic agents such as N-acetylcysteine, Nacystelyn, alginase, mercaptoethanol and the like. Antiviral agents such as ribavirin, gancyclovir, and the like, diamidines such as pentamidine and the like and proteins such as antibodies are also contemplated.

The preferred active agent is a substance or combination of substances that is used for pulmonary prophylactic or rescue therapy, such as a lung surfactant (LS).

Natural LS lines the alveolar epithelium of mature mammalian lungs. Natural LS has been described as a “lipoprotein complex” because it contains both phospholipids and apoproteins that act in conjunction to modulate the surface tension at the lung air-liquid interface and stabilize the alveoli to prevent their collapse. Four proteins have been found to be associated with lung surfactant, namely SP-A, SP-B, SP-C, and SP-D (Ma et al., Biophysical Journal 1998, 74:1899-1907). Specifically, SP-B appears to impart the full biophysical properties of lung surfactant when associated with the appropriate lung lipids. An absence of SP-B is associated with respiratory failure at birth. SP-A, SP-B, SP-C, and SP-D are cationic peptides that can be derived from animal sources or synthetically. When an animal-derived surfactant is employed, the LS is often bovine or porcine derived.

For use herein, the term LS refers to both naturally occurring and synthetic lung surfactant. Synthetic LS, as used herein, refers to both protein-free lung surfactants and lung surfactants comprising synthetic peptides or peptide mimetics of naturally occurring surfactant protein. Any LS currently in use, or hereafter developed for use in RDS and other pulmonary conditions, is suitable for use in the present invention. Current LS products include, but are not limited to, lucinactant (Surfaxin®, Discovery Laboratories, Inc., Warrington, Pa.), poractant alfa (Curosurf®, Chiesi Farmaceutici SpA, Parma, Italy), beractant (Survanta®, Abbott Laboratories, Inc., Abbott Park, Ill.) and colfosceril palmitate (Exosurf®, GlaxoSmithKline, plc, Middlesex, U.K.).

While the methods and systems of this invention contemplate use of active agents, such as lung surfactant compositions, antibiotics, antivirals, mucolytic agents, as described above, the preferred active agent is a synthetic lung surfactant. From a pharmacological point of view, the optimal exogenous LS to use in the treatment would be completely synthesized in the laboratory. In this regard, one mimetic of SP-B that has found to be useful is KL4, which is a 21 amino acid cationic peptide. Specifically the KL4 peptide enables rapid surface tension modulation and helps stabilize compressed phospholipid monolayers. KL4 is representative of a family of LS mimetic peptides which are described for example in U.S. Pat. No. 5,260,273, which is hereby incorporated by reference in its entirety and for all purposes. Preferably the peptide is present within an aqueous dispersion of phospholipids and free fatty acids or fatty alcohols, e.g., DPPC (dipalmitoyl phosphatidylcholine) and POPG (palmitoyl-oleyl phosphatidylglycerol) and palmitic acid (PA). See, for example, U.S. Pat. No. 5,789,381 the disclosure of which is incorporated herein by reference in its entirety and for all purposes).

In a preferred embodiment, the LS is lucinactant or another LS formulation comprising the synthetic surfactant protein KLLLLKLLLLKLLLLKLLLL (KL4). The preferred LS, lucinactant, is a combination of DPPC, POPG, palmitic acid (PA) and the KL4 peptide. In some embodiments, the drug product is formulated at concentrations of, for example, 10, 20, and 30 mg/ml of phospholipid content. In other embodiments, the drug product is formulated at greater concentrations, e.g, 60, 90, 120 or more mg/ml phospholipid content, with concomitant increases in KL4 concentration.

Preferably when surfactants are utilized in practicing the method of the present invention they are selected to be present in an amount sufficient to effectively modulate the surface tension of the liquid/air interface of the epithelial surface to which they are applied.

This invention contemplates the use of other cationic peptides beyond KL4 surfactant. Preferably, cationic peptides consist of at least about 10, preferably at least 11 amino acid residues, and no more than about 60, more usually fewer than about 35 and preferably fewer than about 25 amino acid residues.

Many cationic peptides have been disclosed in the art. See, for example, U.S. Pat. Nos. 5,164,369, 5,260,273, 5,407,914; and 6,613,734, each of which is hereby incorporated by reference in its entirety and for all purposes. Examples of cationic peptides include KLLLLKLLLLKLLLLK (KL4, SEQ ID NO:1), DLLLLDLLLLDLLLLDLLLLD (DL4, SEQ ID NO:2 ), RLLLLRLLLLRLLLLRLLLLR (RL4, SEQ ID NO:3), RLLLLLLLLRLLLLLLLLRLL (RL8, SEQ ID NO:4), RRLLLLLLLRRLLLLLLLRRL (R2L7, SEQ ID NO:5), RLLLLCLLLRLLLLLCLLLR (SEQ ID NO:6), RLLLLLCLLLRLLLLCLLLRLL (SEQ ID NO:7), and RLLLLCLLLRLLLLCLLLRLLLLCLLLRDLLLDLLLDLLLDLLLDLLLD (SEQ ID NO:8), and polylysine, magainans, defensins, iseganan, histatin and the like. Preferably, the cationic peptide is the LS mimetic, KL4.

“LS mimetic peptides” as used herein refers to polypeptides with an amino acid residue sequence that has a composite hydrophobicity of less than zero, preferably less than or equal to −1, more preferably less than or equal to −2. The composite hydrophobicity value for a peptide is determined by assigning each amino acid residue in a peptide its corresponding hydrophilicity value as described in Hopp, et al. Proc. Natl. Acad. Sci., 78: 3824-3829 (1981), which disclosure is incorporated by reference. For a given peptide, the hydrophobicity values are summed, the sum representing the composite hydrophobicity value.

These hydrophobic polypeptides perform the function of the hydrophobic region of the SP18, a known LS apoprotein. SP-18 is more thoroughly described in Glasser, et al., Proc. Natl. Acad. Sci., 84:4007-4001 (1987), which is hereby incorporated by reference. In a preferred embodiment, the amino acid sequence mimics the pattern of hydrophobic and hydrophilic residues of SP18.

A preferred LS mimetic peptide includes a polypeptide having alternating hydrophobic and hydrophilic amino acid residue regions and is characterized as having at least 10 amino acid residues represented by the formula:
(ZₐU_{b})_{c}Z_{d}
Z and U are amino acid residues such that at each occurrence Z and U are independently selected. Z is a hydrophilic amino acid residue, preferably selected from the group consisting of R, D, E and K. U is a hydrophobic amino acid residue, preferably selected from the group consisting of V, I, L, C, Y, and F. The letters, “a,” “b,” “c” and “d” are numbers which indicate the number of hydrophilic or hydrophobic residues. The letter “a” has an average value of about 1 to about 5, preferably about 1 to about 3. The letter “b” has an average value of about 3 to about 20, preferably about 3 to about 12, most preferably, about 3 to about 10. The letter “c” is 1 to 10, preferably, 2 to 10, most preferably 3 to 6. The letter “d” is 1 to 3, preferably 1 to 2.

By stating that the amino acid residue represented by Z and U is independently selected, it is meant that each occurrence, a residue from the specified group is selected. That is, when “a” is 2, for example, each of the hydrophilic residues represented by Z will be independently selected and thus can include RR, RD, RE, RK, DR, DD, DE, DK, etc. By stating that “a” and “b” have average values, it is meant that although the number of residues within the repeating sequence (ZaUb) can vary somewhat within the peptide sequence, the average values of “a” and “b” would be about 1 to about 5 and about 3 to about 20, respectively.

Exemplary preferred polypeptides of the above formula are shown in the Table of LS Mimetic Peptides.

Table of LS Mimetic Peptides

| | SEQ. | |
|---|---|---|
| Designation¹ | ID. NO. | Amino Acid Residue Sequence |
| DL4 | 4 | DLLLLDLLLLDLLLLDLLLLD |
| RL4 | 5 | RLLLLRLLLLRLLLLRLLLLR |
| RL8 | 6 | RLLLLLLLLRLLLLLLLLRLL |
| RL7 | 7 | RRLLLLLLLRRLLLLLLLRRL |
| RCL1 | 8 | RLLLLCLLLRLLLLCLLLR |
| RCL2 | 9 | RLLLLCLLLRLLLLCLLLRLL |
| RCL3 | 10 | RLLLLCLLLRLLLLCLLLRLLLLCLLLR |
| KL4 | 1 | KLLLLKLLLLKLLLLKLLLLK |
| KL8 | 2 | KLLLLLLLLKLLLLLLLLKLL |
| KL7 | 3 | KKLLLLLLLKKLLLLLLLKKL |

¹The designation is an abbreviation for the indicated amino acid residue sequence.

Examples of phospholipids useful in the compositions delivered by the invention include native and/or synthetic phospholipids. Phospholipids that can be used include, but are not limited to, phosphatidylcholines, phospatidylglycerols, phosphatidylethanolamines, phosphatidylserines, phosphatidic acids, and phosphatidylethanolamines. Exemplary phospholipids include dipalmitoyl phosphatidylcholine (DPPC), dilauryl phosphatidylcholine (DLPC) C12:0, dimyristoyl phosphatidylcholine (DMPC) C14:0, distearoyl phosphatidylcholine (DSPC), diphytanoyl phosphatidylcholine, nonadecanoyl phosphatidylcholine, arachidoyl phosphatidylcholine, dioleoyl phosphatidylcholine (DOPC) (C18:1), dipalmitoleoyl phosphatidylcholine (C16:1), linoleoyl phosphatidylcholine (C18:2)), dipalmitoyl phosphatidylethanolamine (DPPE), dioleoylphosphatidylethanolamine (DOPE), dioleoyl phosphatidylglycerol (DOPG), palmitoyloleoyl phosphatidylglycerol (POPG), distearoylphosphatidylserine (DSPS) soybean lecithin, egg yolk lecithin, sphingomyelin, phosphatidylserines, phosphatidylglycerols, phosphatidylinositols, diphosphatidylglycerol, phosphatidylethanolamine, and phosphatidic acids, Egg phosphatidylcholine (EPC)

Examples of fatty acids and fatty alcohols useful in these mixtures include, but are not limited to, palmitic acid, cetyl alcohol, lauric acid, myristic acid, stearic acid, phytanic acid, dipamlitic acid, and the like. Preferably, the fatty acid is palmitic acid and preferably the fatty alcohol is cetyl alcohol.

The terms “medium” or “media” refer to both aqueous and non-aqueous media. The preferred medium is chosen so as not cause any adverse effect on the biological activity of the active agent being delivered.

Preferably, a non-aqueous medium can include, for example, hydrogen-containing chlorofluorocarbons, fluorocarbons and admixtures thereof. To provide some adjunctive respiratory support, and to provide efficient lung filling in the degassed state, the perfluorocarbon liquid should have an oxygen solubility greater than about 40 ml/100 ml. Representative perfluorocarbon liquids include FC-84, FC-72, RM-82, FC-75 (3M Company, Minneapolis, Minn.), RM-101 (MDI Corporation, Bridgeport, Conn.), dimethyladamantane (Sun Tech, Inc.), trimethylbicyclononane (Sun Tech, Inc.), and perfluorodecalin (Green Cross Corp., Japan).

Preferably, when an aqueous medium is employed, the medium is a water-containing liquid. Suitable media include isotonic ionic solutions preferably buffered to within 1 pH unit of physiologic pH (7.3). The medium should be free of pathogens and other deleterious materials and can be composed of pure water but also optionally can include up to about 20% by volume and preferably up to about 5% of nontoxic organic liquids such as oxy-group containing liquids such as alcohols, esters, ethers, ketones and the like. In selecting organic components it is important to avoid materials which are likely to give rise to undesired reactions such as intoxication, sedation, and the like. Preferably, the medium is saline or tromethamine buffer.

The present invention provides methods of delivering an aerosolized active agent to a patient. Typically, such methods include a step of generating a stream of particles with an aerosol generator to produce the aerosolized active agent. In accordance with some embodiments, the methods of the present invention include a step of impacting the aerosolized active agent with a stream of gas. In embodiments wherein a stream of gas is employed, the aerosolized active agent will preferably be impacted by the gas in a uniform manner, for example, in a substantially radially symmetric manner. By impacting the aerosolized active agent, for example, in a substantially radially symmetric manner, the gas is able to direct the aerosolized active agent to the delivery outlet.

In some embodiments, the stream of gas is part of a conditioning system. The conditioning system employs the gas, now referred to as a conditioning gas, to direct the aerosol, for example, to the inspiratory gas flow. In some embodiments, the conditioning gas will not only modulate the flow of aerosolized active agent but will alter one or more characteristics of the active agent mixture. For example, in some embodiments, the conditioning gas will alter the characteristics of at least a portion of the aerosol generated by the aerosol generator to produce a second aerosol. An example of the characteristics of the aerosol that can be altered includes aerosol particle size and ratio of active agent to medium. While not wishing to be bound by any particular theory, it is believed that by decreasing the size of the particle, deposition on ex vivo sites can be decreased because the chaotic flow regimes are minimized. It is also believed that the conditioning gas can, in some occasions, evaporate off a portion of the medium present in the particles. Accordingly, the conditioning gas can, in some embodiments, shape, bound and/or direct the aerosol flow and in so doing can create a buffer zone between the aerosol and the physical walls of the delivery apparatus

Preferably, the stream of gas or conditioning gas refers to air and other fabricated gaseous formulations containing air, oxygen gas, nitrogen gas, helium gas, nitric oxide gas and combinations thereof (e.g., heliox or “trimix” of helium, oxygen and nitrogen), as would be understood by one of skill in the art of respiratory therapy. Preferably, the gas is a formulation of air and oxygen gas, wherein the oxygen content is varied from about 20% to about 100% of the total gas composition. The amount of oxygen in the gas formulation is readily determined by the attending clinician.

The term “sheath gas” and “conditioning air” is used synonymously with “conditioning gas”.

The terms “bounding, shaping, and directing” and “shape, bound and direct” as used herein refer to the conditioning performed by the conditioning gas to the stream of particles in the aerosol. This is most clearly illustrated in FIG. 8. Specifically, the stream of particles in the aerosol are contacted with the conditioning gas. The conditioning gas can, in some instances, shape the stream of particles into a more condensed, focused flow (i.e., provide directional coherence to the aerosol stream of particles) bounded by conditioning gas. As shown in FIG. 8, this shaped, bounded flow of particles is directed to the delivery apparatus. One of ordinary skill in the art would readily appreciate that the effect(s) of impacting an aerosol with “conditioning gas” can vary depending on the characteristics of the aerosol, the equipment configurations, and operating parameters—thus, the conditioning illustrated in FIG. 8 is exemplary only and is not intended to limit the scope of the appended claims.

In some embodiments, a significant fraction of the aerosol is conditioned by the conditioning gas. A significant fraction refers to more than about 10% of the aerosol; preferably more than about 25%; more preferably more than about 50%; and still more preferably more than about 90%.

Preferably, the gas is added at a flow rate so as not to create a turbulent gas flow. Preferably, the volume per unit of time of conditioning gas flow is from about 0.1 to about 6 l/min and is dependent on the patient. The flow is optimized based on the amount of aerosol that is generated from the nebulizer, and more particularly is optimized to a rate that the aerosol deposition in the conditioner and other parts of the delivery tract can be minimized as well as minimizing dilution of the aerosol.

In embodiments wherein the conditioning gas is used to evaporate off a portion of the medium present in the particles, it is believed that the conditioning gas may accelerate the evaporation of medium from the particles in the aerosol as the particles move from the nebulizer where they are generated to the point of delivery to the patient. This evaporation can be expedited when the conditioning gas is heated and/or presented at a relatively low moisture (humidity) level. Preferably, the temperature of the conditioning gas is about 37 to about 50° C. and more preferably about 37 to about 42° C. Preferably, the conditioning gas has a relative humidity at 37° C. of less than about 60%, more preferably less than about 20%, and even more preferably less than about 5% relative humidity. Alternatively, the conditioning gas can have a higher relative humidity, including up to 100% relative humidity.

In some aspects of the invention the conditioning gas evaporates the particles so that particles are substantially free of the medium. Substantially free means that the aerosol being delivered does not contain a significant amount of medium.

As discussed in detail below, many embodiments of the invention involve delivery of the aerosolized active agent in conjunction with another noninvasive pulmonary respiratory therapy involving the administration of positive airway pressure. The term “noninvasive pulmonary respiratory therapy” refers to respiratory therapy which does not use mechanical ventilation and can include CPAP, bilevel positive airway pressure (BiPAP), synchronized intermittent mandatory ventilation (SIMV), and the like. The employment of such therapies involves the use of various respiratory gases, as would be appreciated by the skilled artisan. Respiratory gases used for noninvasive pulmonary respiratory therapy are sometimes referred to herein as “CPAP gas,” “CPAP air,” “ventilation gas,” “ventilation air,” or simply “air.” However, those terms are intended to include any type of gas normally used for noninvasive pulmonary respiratory therapy, including but not limited to gases and gaseous combinations listed above for use as the conditioning gas. In certain embodiments, the gas used for noninvasive pulmonary respiratory therapy is the same as the conditioning gas. In other embodiments, the respective gases are different from one another.

In certain embodiments, the pulmonary delivery methods of this invention are employed in conjunction with CPAP. It has been shown that use of CPAP allows for an increase in functional residual capacity and improved oxygenation. The larynx is dilated and supraglottic airway resistance is normal. There is also an improvement of the synchrony of respiratory thoracoabdominal movements and enhanced Hering-Breuer inflation reflex following airway occlusion. CPAP has been shown to be useful in treating various conditions such as sleep apnea, snoring, ARDS, IRDS, and the like.

In order to effect administration of CPAP, a pressure source and a delivery device or delivery apparatus are required. CPAP-producing airflow is typically generated in the vicinity of the nasal airways by converting kinetic energy from a jet of fresh humidified gas into a positive airway pressure. A continuous flow rate of breathing gas of about 5 to about 12 liters/minute generates a corresponding CPAP of about 2 to about 10 cm H₂O. Various modifications may be applied to the CPAP system which include sensors that can individualize the amount of pressure based on the patient's need.

Typically, flow rates and pressures suitable for achieving CPAP are based upon the characteristics of the patient being treated. Patients subject to treatment by the methods of the present invention can be neonatal infants, infants, juveniles and adults. Typically a neonatal infant is an infant born prematurely or otherwise, under 4 weeks old. Infants typically refer to those older than 4 weeks old but under 2 years old. Juveniles refer to those individuals older than 2 years old but under 11 years old. Adults are older than 11 years old.

Suitable flow rates and pressures can be readily calculated by the attending clinician. The present invention encompasses the use of a variety of flow rates for the ventilating gas, including low, moderate and high flow rates. Alternatively, the aerosol can be supplied without added positive pressure, i.e., without CPAP as a simultaneous respiratory therapy.

Preferably, the CPAP-generating air flow being delivered to the patient has a moisture level which will prevent unacceptable levels of drying of the lungs and airways. Thus, the CPAP-generating air is often humidified by bubbling through a hydrator, or the like to achieve a relative humidity of preferably greater that about 70%. More preferably, the humidity is greater than about 85% and still more preferably 98%.

A suitable source of CPAP-inducing airflow is the underwater tube CPAP (underwater expiratory resistance) unit. This is commonly referred to as a bubble CPAP.

Another preferred source of pressure is an expiratory flow valve that uses variable resistance valves on the expiratory limb of CPAP circuits. This is typically accomplished via a ventilator.

Another preferred source is the Infant Flow Driver or “IFD” (Electro Medical Equipment, Ltd., Brighton, Sussex, UK). IFD generates pressure at the nasal level and employs a conventional flow source and a manometer to generate a high pressure supply jet capable of producing a CPAP effect. It is suggested in the literature that the direction of the high pressure supply jet responds to pressures exerted in the nasal cavity by the patient's efforts and this reduces variations in air pressure during the inspiration cycle.

Other CPAP systems including those that contain similar features to systems just discussed are also contemplated by the present invention.

The aerosol stream generated in accordance with the present invention is preferably delivered to the patient via a nasal delivery device which may involve, for example masks, single nasal prongs, binasal prongs, nasopharyngeal prongs, nasal cannulae and the like. The delivery device is chosen so as to minimize trauma, maintain a seal to avoid waste of aerosol, and minimize the work the patient must perform to breathe. Preferably, binasal prongs are used.

The aerosol stream can also be delivered orally. Preferred oral delivery interfaces include masks, cannulae, and the like.

The methods, systems, and devices of the present invention deliver aerosolized active agents to the lungs. In some embodiments, the aerosolized active agent is conditioned before delivery, i.e., impacted with a conditioning gas or other conditioning means.

As illustrated schematically in FIG. 1, the invention employs a mixture of active agent in a medium. This mixture can be formed by adding the active agent and the medium into mixing vessel 12 via lines 10 and 14, respectively. The order of addition is not critical. In this example, the active agent and the medium are mixed with the mixing blade 13 to provide the desired substantially homogeneous mixture. The medium and active agent are added in sufficient amounts to provide a concentration that will be effective when delivered to the patient via the present improved aerosolization process. They may be mixed batchwise or in a continuous process.

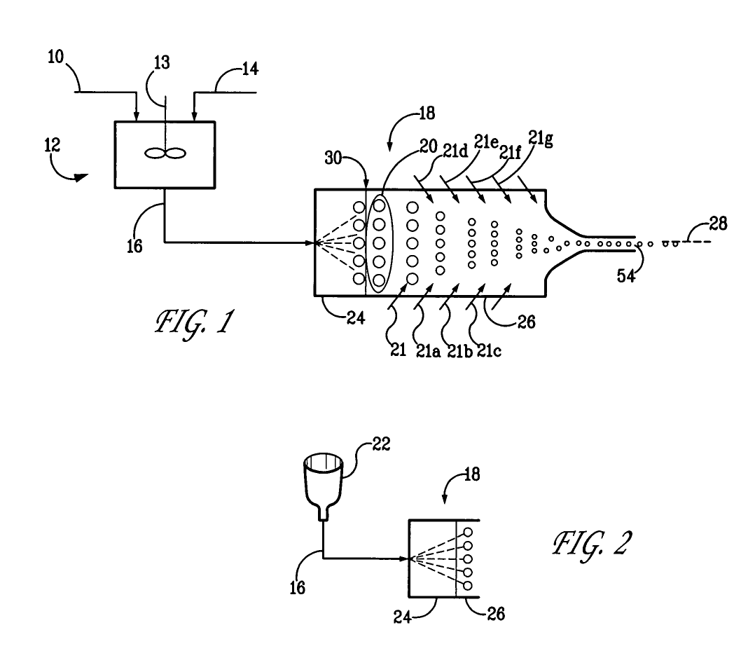

In an alternative embodiment, the medium and active agent are premixed. As depicted in FIG. 2, the premix is present in vessel 22.

The mixture of active agent and medium is passed to conditioner 18 via line 16 and then treated as described below.

Most aerosol particles carry some electric charge that could cause particle repulsion, and thus deposition. As such, in an alternative embodiment, the nebulizer 24 and the various components of the conditioner discussed below can be coated with a material that could reduce particle deposition and/or repulsion. This material is preferably wettable and can also act as a static control agent to the aerosol. Alternatively, the material may be blended with the additive and produced via extrusion compounding.

Another approach to reducing deposition and/or repulsion would be to mix the aerosol with high concentration of bipolar ions produced by corona discharge or radiation. The aerosol neutralizer can be placed downstream of the nebulizer 24 or mixed with the conditioning gas prior to the conditioning gas entering into the conditioner as described below.

The mixture is fed via line 16 into conditioner 18. The operation of conditioner 18 is depicted in FIGS. 1 and 8 and reference should be made to both. Conditioner 18 includes a nebulizer (aerosol generator) 24 in fluid-tight communication with a conditioning vessel 26. In one embodiment, the aerosol generator is an ultrasonic nebulizer or vibrating membrane nebulizer or vibrating screen nebulizer. Typically jet nebulizers are not employed although the present methods can be adapted to all types of nebulizers or atomizers. In one embodiment, the aerosol generator is an Aeronebg Professional Nebulizer (Aerogen Inc., Mountain View, Calif., USA). Nebulizer 24 generates a high density, disorganized (nonconditioned) stream of particles of the mixture. The size of the aerosol particles is not critical to the present invention. A representative non-limited list of particle MMAD ranges include from about 0.5 to about 10 microns, from about 1 to about 10 microns, from about 0.5 to about 8 microns, from about 0.5 to about 6 microns, from about 0.5 to about 3 microns, and from about 0.5 to about 2 microns in size. Aerosol particles having a MMAD of less than 0.5 microns or greater than 10 microns are equally contemplated by the present invention.

In another embodiment, the aerosol generator is a capillary aerosol generator, an example of which is the soft-mist generator available from Chrysalis Technologies, Richmond, Va. (T. T. Nguyen, K. A. Cox, M. Parker and S. Pham (2003) Generation and Characterization of Soft-Mist Aerosols from Aqueous Formulations Using the Capillary Aerosol Generator, J. Aerosol Med. 16:189).

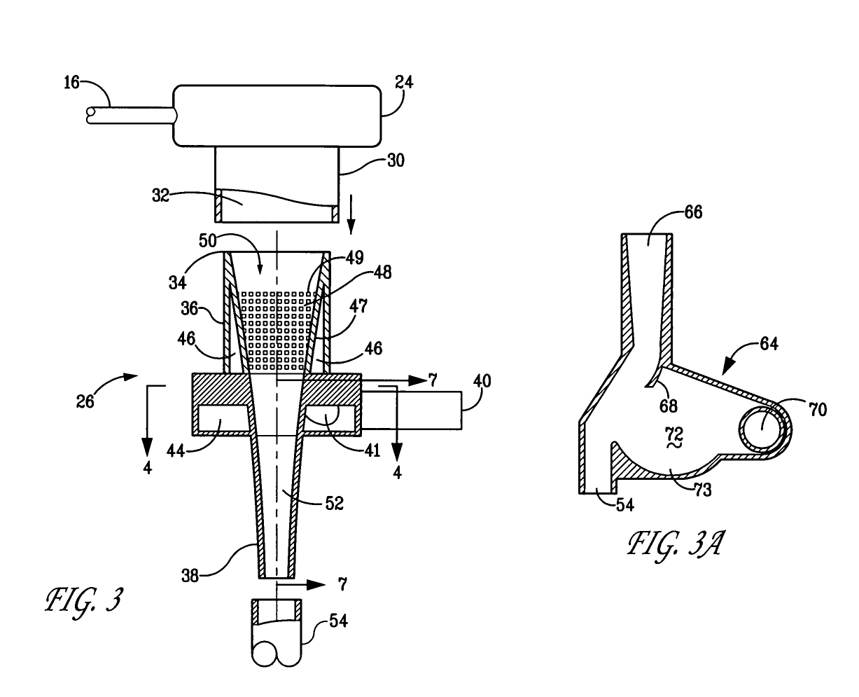

Some embodiments of the invention include the use of a stream of gas or conditioning gas, while other embodiments do not, as will be apparent from the drawings and their description herein. In some embodiments comprising use of a conditioning gas, unconditioned aerosol 20 is passed to conditioning vessel 26 via opening 50 (see FIG. 3), where the aerosol is conditioned with the conditioning gas which is depicted in FIG. 1 as gas streams 21 though 21g. As FIGS. 1 and 8 illustrate, the conditioning gas flows 21-21g can, in some embodiments, evaporate medium from the particles preferably accelerating their reduction in size from a first MMAD toward a second, smaller MMAD and, as a consequence, the smaller droplets will have a greater chance of transiting the delivery system and being delivered to the lungs. As the particles reduce in size the probability that they will be intercepted by surfaces is diminished as their inertia is reduced. The conditioning gas can, in some embodiments, also causes the stream of particles to be bounded, shaped, and directed into a more focused coherent stream 28 (see FIG. 8) in conditioning vessel 26. Note that the present invention includes some methods, embodiments, and devices wherein the aerosolized active agent is essentially unchanged from the aerosol generator to the point of delivery to a patient.

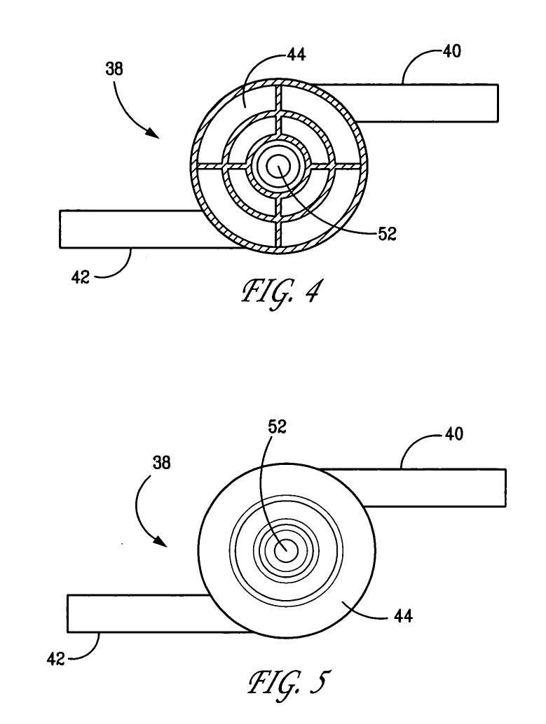

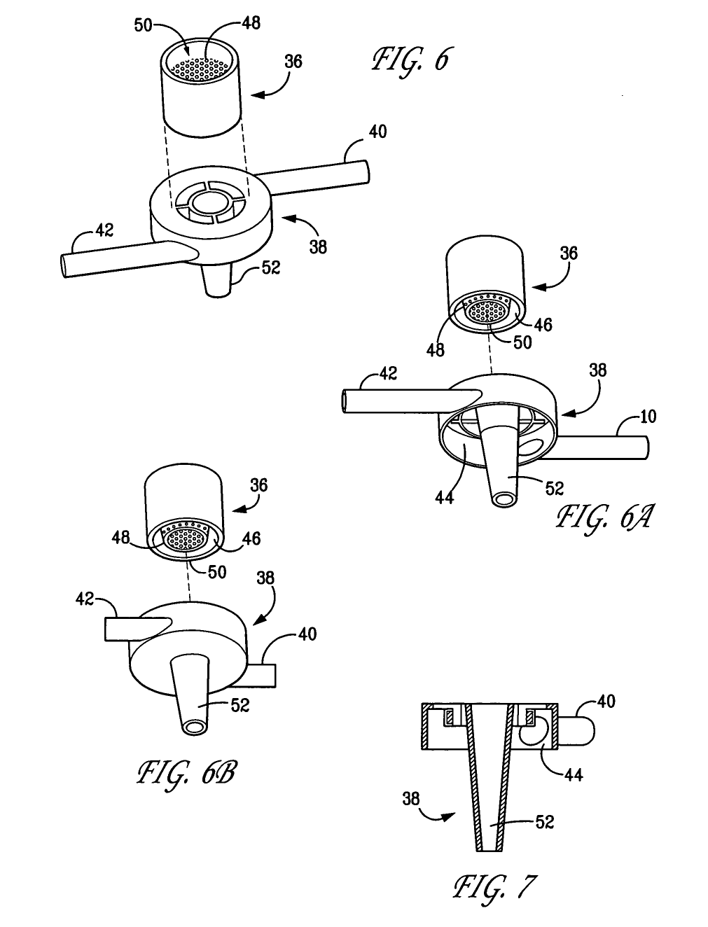

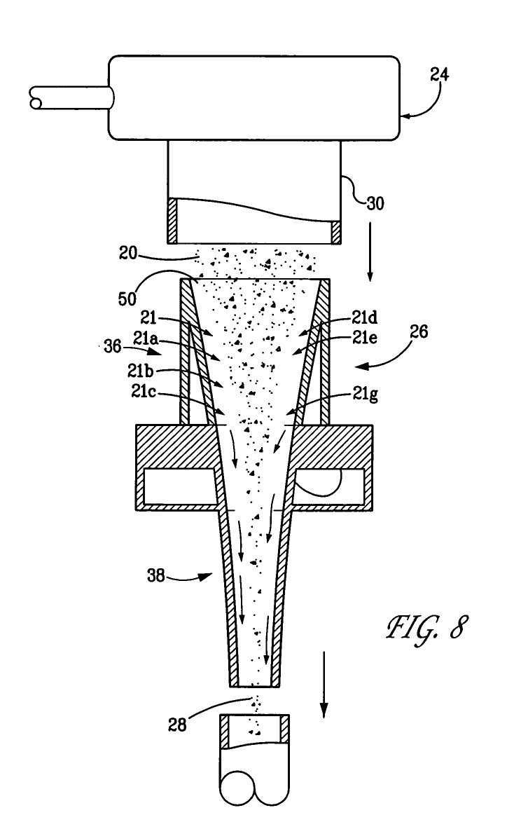

As shown in FIGS. 3 and 8, in one embodiment the nebulizer 24 includes an outlet sleeve 30 having an internal dimension 32 which allows it to achieve a tight slip fit seal over the inlet body 34 of conditioning vessel 26. Based upon the specific nebulizer employed, the junction between the nebulize employed, the junction between the nebulizer 24 and the conditioning vessel may be modified accordingly. Although not shown in this configuration, nebulizer 24 may be spaced apart from conditioning vessel 26 and connected via flexible tubing or the like. As best shown in FIG. 3, conditioning vessel 26 is comprised of two parts, conditioning gas inlet unit 36 and conditioned flow nozzle 38. Details of these two units are illustrated in FIGS. 3, 4, 5, 6, 6A, 6B and 7. The conditioning gas stream enters conditioning gas unit inlet 38 via inlets 40 and 42 line having opening 41 which delivers the conditioning gas flow into chamber 44. Preferably the flow rate is set to ensure a non-turbulent flow. As already discussed, the conditioning gas will, in some cases have had its temperature adjusted and its moisture level monitored and most likely modified so as to give rise in suitable levels of evaporation of medium from the particles 20 as they contact the conditioning gas flow. Apparatus to accomplish this temperature and moisture level adjustment in patient ventilation settings are known in the art and are not depicted in these drawings.

As depicted in FIG. 3 the conditioning gas circulates in chamber 44 and up into adjacent chamber 46 where it surrounds the aerosol flow zone 50 defined by tapered conical wall 47. Wall 47 includes a region 48 which contains a plurality of openings 49. In FIG. 3 these openings are depicted as a series of holes surrounding the flow zone 50 defined by wall 47. The conditioning gas from chamber 46 then passes through openings 49 in region 48. While the openings 49 in region 48 are depicted in a perforated design, this invention contemplates other designs that allow for uniform distribution (i.e., preferably radially symmetric flow) of sheath gas such as slits and the like. The conditioning gas flowpaths through openings 49 are those schematically represented as flows 21-21g in FIGS. 1 and 8. As shown in those Figs. the flow paths of conditioning gas are calibrated preferably to provide a nonturbulent flow regime which exits from the aerosol flow zone defined by tapered wall 47 out through nozzle 52. The aerosol of particles of the active agent-media mixture is bounded, shaped, and directed by the conditioning gas and is carried out of the conditioning gas unit 36 and out through nozzle 52 as a coherent flow of particles having a reduced size as compared to particles 20, originally generated by nebulizer 24.

It will be appreciated that the conditioning gas generator will have capabilities to recognize when the systems of this invention are over-pressurized and will adjust the conditioning gas flow appropriately.

The conditioning gas delivered through openings 49 acts as a buffer between the wall 47 of flow zone 50 and the unconditioned aerosol and thus reduces clogging in nozzle 52 due to accumulation of aerosol solids or condensed liquids on wall 47. This buffer-effect is continued through the delivery device, for example trough nozzle 52.

In some embodiments, the conditioning gas creates a conditioned aerosol not only by bounding, shaping and directing the aerosol's flow but also by evaporating liquid medium out of the particles 20 and thus reducing the average particle size (MMAD) of the particles present in the aerosol. It is to be recognized that the evaporation of liquid medium leads to a change in the volume of the particles and particle volume change is a function of the cube of the particle diameter change.

If desired, this aerosol flow with its conditioning gas can be delivered directly to the oral or nasal pathway with well-known devices that include for example only, masks, single nasal prongs, binasal prongs, nasopharyngeal prongs, nasal cannulae and the like. An embodiment of the invention shown in FIG. 12 illustrates the use of binasal prongs 100. FIG. 12A shows an exemplary embodiment of the present invention with nasal prongs 100 inserted into the nares of an infant (FIG. 12A's reference numbers are described below). The device is chosen based upon the disorder being treated and the patient. Preferably, the device chosen maintains a seal between the device and the patient to avoid loss of aerosol product and, importantly to maintain continuous positive air pressure.

When setting the flow rate of the conditioning gas and the flow rate out of nozzle 52 one of skill in the art would also take into consideration the nature of the patient being treated and the route of administration (nasal versus oral). Typical flow rates of nozzle 52 will be readily determined by the attending clinician.

Typically, the conditioning gas and conditioned aerosol are delivered to the patient at a delivery temperature of about 20 to about 40° C. The delivery temperature refers to the temperature at which the aerosol and air are received by the patient. As such, the conditioning gas typically enters the conditioner at about 0 to about 25° C. above the delivery temperature. Preferably, the conditioning gas has an initial temperature of about 37 to about 45° C.

In addition to the administration methods just described, this invention contemplates delivering the conditioned aerosol to a patient while simultaneously administering other forms of noninvasive respiratory therapy. Preferably, the therapy is CPAP. Still more preferably, the therapy utilizes bubble CPAP or even more preferably some form of synchronized therapy wherein the positive pressure is varied in response to inspiratory maneuvers by the patient.

When delivering the aerosol simultaneously with the CPAP-producing airflow, it is desirable to minimize the contact of the conditioned aerosol with the CPAP-producing airflow prior to delivery to the patient. Problems may arise when the two components are extensively mixed prior to delivery. Mainly, contact of the two flows can, in some instances, lead to a decreased amount of aerosol that is delivered to the patient due to the dilution of the aerosol with the CPAP-producing airflow.

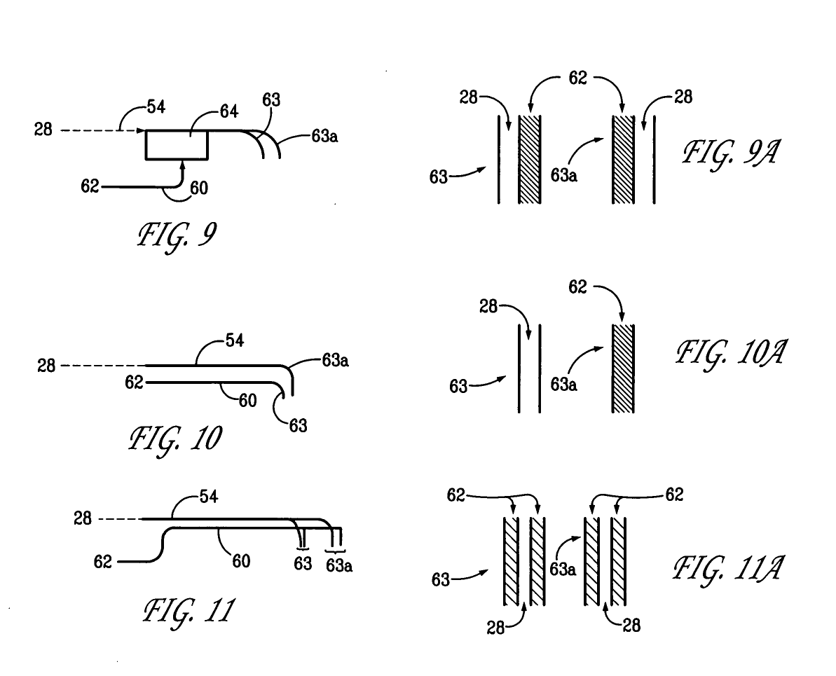

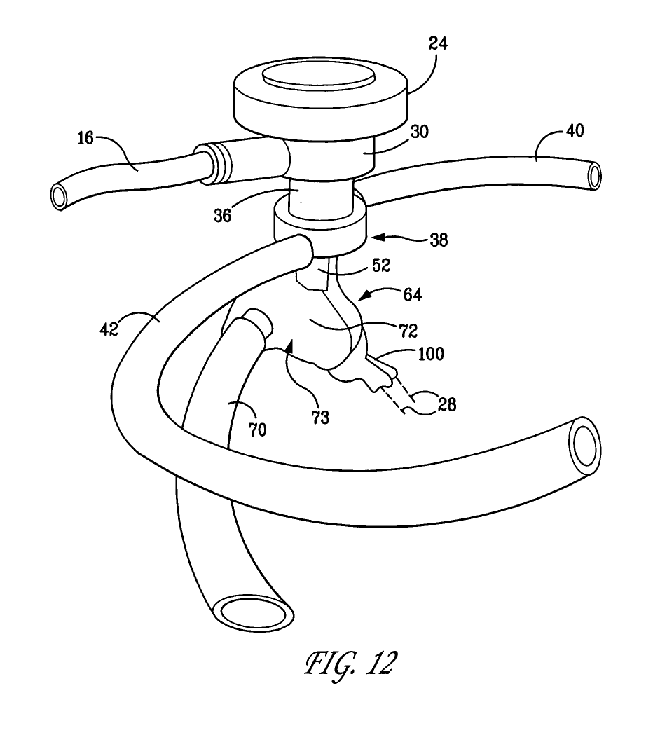

To that end, this invention contemplates several approaches to the simultaneous delivery of a CPAP-producing airflow and a conditioned aerosol designed to minimize premature contact of the CPAP-producing airflow with the conditioned aerosol. These are represented schematically in FIGS. 9 through 11 and 9A through 11A. While these depicted embodiments describe nasal prong designs, it is contemplated that based on the principles of the designs, only minor modifications would need to be made to effect similar delivery via a nasal mask or for an oral delivery device. For example, when the conditioned aerosol and CPAP-generating airflow are being delivered orally, suitable modifications may be made to the oral delivery device to accommodate two separate lines in a manner similar to the nasal prongs.

For the following embodiments, a CPAP generator (not shown) generates a suitable flow of CPAP-producing air 62 delivered via line 60. Line 54 delivers contains conditioned aerosol 28.

In one embodiment, the CPAP generator and the conditioning gas generator are the same ventilator-like machine and a flow-splitter is employed or a ventilator-like machine that has two gas outlet ports. The use of a flow-splitter allows for the CPAP gas and the conditioning gas to have the same gas composition, temperatures, humidity and the like of the flows to be altered independently of one another.

In another embodiment, the CPAP-producing airflow and the conditioning gas are heated by independent heating sources to allow the CPAP-producing airflow to be both heated and humidified, while the conditioning gas is only heated. It should be noted that the conditioning gas will become slightly humidified upon contact with the aerosol.

This invention also contemplates employing an isolation valve or other mechanism that can be used to provide a complete sealed environment that will allow positive airway pressure to be maintained while aerosol is not delivered. In other words, the valve can be used to maintain continuous operation of CPAP with or without aerosol delivery. Situations when aerosol is not delivered include changing nebulizer, cleaning the conditioner or stopping the surfactant therapy altogether when the efficacy is reached.

In one embodiment, shown in FIGS. 9 and 9A conditioned aerosol 28 and CPAP 62 are mixed immediately prior to delivery to the patient. The CPAP airflow 62, delivered via line 60 and the conditioned aerosol delivered via line 54 are mixed in mixer 64 just prior to delivery to the patient. FIG. 9A is a cross-sectional view of the same. Conditioned aerosol 28 and CPAP 62 are delivered as a mixture to the patient via both nasal prongs 63 and 63A.

FIG. 3a illustrates one embodiment of the mixer or fluid flow connector 64 referenced in FIG. 9. Mixer or fluid flow connector 64 includes an inlet 66 designed to seal and mate with nozzle 52 of the aerosol generator/conditioner shown in, for example FIG. 1. The flow of aerosol 54 produced in the generator/conditioner where it enters chamber 72. A CPAP-inducing flow of gas is fed into chamber 72 via CPAP airflow feed line 70. There is typically an outlet in-line with line 70. The combined flows pass through orifice 54 to nasal prongs or other like delivery devices as previously discussed. Chamber 72 is optionally equipped with baffles such as 68 so as to direct the aerosol to the outlets and to minimize premature contact between the conditioned aerosol and the CPAP-producing airflow. In an alternative embodiment, baffles are not employed. Chamber 72 is further designed to minimize turbulence and mixing between the two flows. Chamber 72 is also designed to minimize the likelihood that solids or condensed liquids will occlude the delivery apparatus like nasal prongs or enter the patient's airways and may include for example a solid/liquid trap 73 which acts as a collection and/or extraction repository. Any material that is collected in the trap 73 may be extracted and recycled but more commonly is discarded. Alternatively, a port may be incorporated that allows for liquid removal.

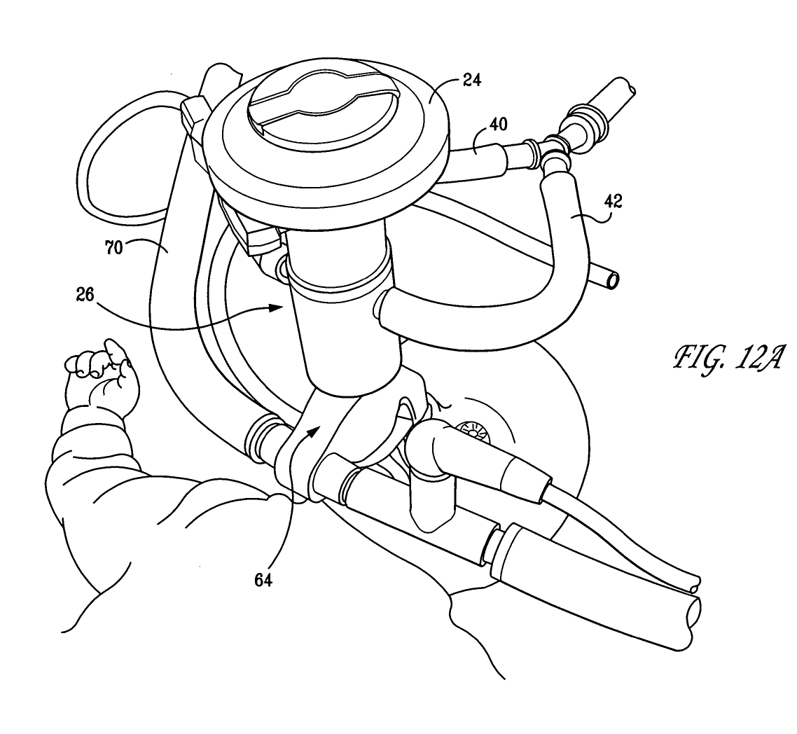

In another embodiment shown in FIGS. 10 and 10A, conditioned aerosol 28 and CPAP-producing airflow 62 are not mixed prior to delivery of the patient, but instead are delivered separately via lines 54 and 60 respectively to separate nasal prongs 63 and 63A.

In yet another embodiment shown in FIGS. 11 and 11A, the conditioned aerosol 28 fed through line 54 and the CPAP-producing airflow 62 fed through line 60 are delivered separately with minimal mixing and with the CPAP-producing airflow coaxially surrounding the conditioned aerosol stream. It will be appreciated that this is essentially the same configuration that is present between the conditioning air flow and the initial aerosol. To that end, one might use a device similar to the conditioning unit to add extra coaxial CPAP-producing air to the flow. Alternatively, in some cases, it might be possible to increase the flow of conditioning gas to a point that it would be able to induce a CPAP condition in the patient.

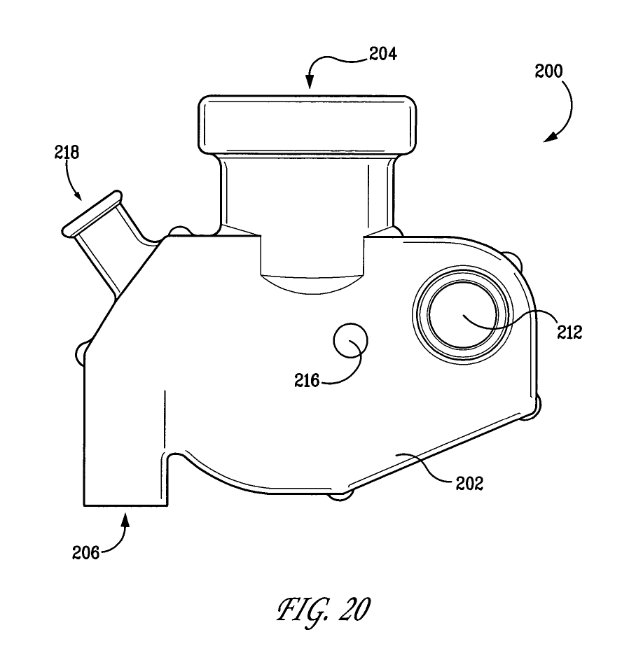

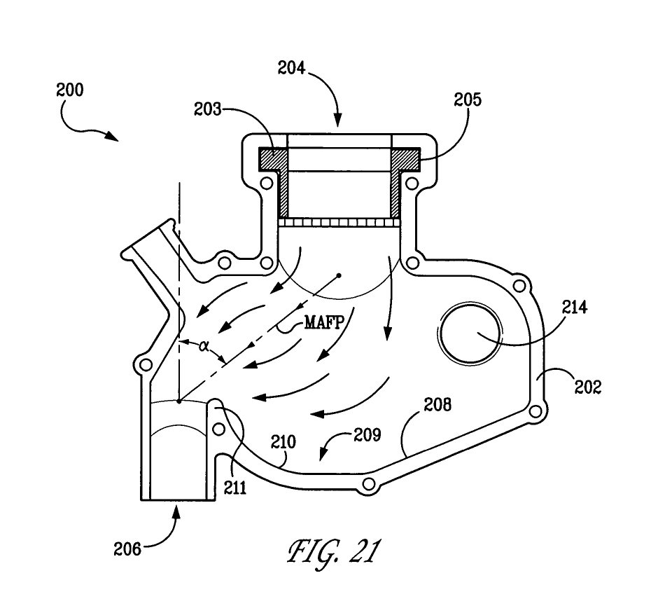

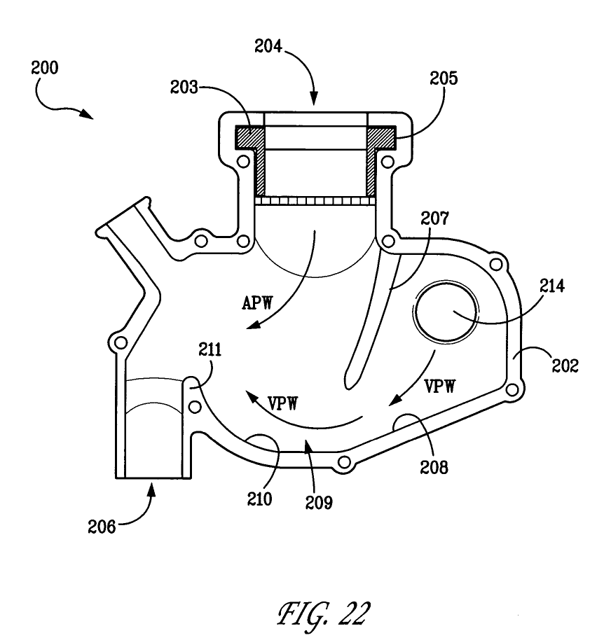

Referring now to FIGS. 18-21, an exemplary fluid flow connector 200 is shown substantially in the form of an enclosed chamber 202 having a series of ports (some of which are optional) disposed therein. Connector 202 is referred elsewhere in this specification as a “mixer,” or a “prong adapter” since nasal prongs can optionally be connected to the chamber. Chamber 202 includes an aerosol inlet 204 for receiving an active agent that has been aerosolized by an aerosol generator (not shown) that can be connected directly or indirectly to fluid flow connector 200. Any number of devices can be inserted into aerosol inlet 204 for supplying the aerosolized active agent, such as, for example, tubing, tubing fittings (e.g., a nipple), or a mating connector. Aerosol inlet 204 may employ a valve. By way of example only, a cross slit valve 203 can be seated in annular channel 205 (see FIG. 21). The aerosolized active agent exits chamber 202 through a delivery outlet 206, which is preferably in fluid communication with a pair of nasal prongs. The delivery outlet can also be configured for connection with a mask, a diffuser, or any other device known by the skilled artisan that is placed near a patient's mouth and/or nose for inhalation of the aerosolized active agent. In some embodiments, the delivery outlet will be indirectly connected to a pair of nasal prongs or other device for inhalation of the aerosolized active agent. For example, the delivery outlet 206 of the fluid flow connecter 200 can, in some embodiments, communicate the aerosolized active agent to another device or conduit that is in fluid communication with, for example, a pair of nasal prongs but that is not necessarily configured to collect deposits associated with the aerosolized active agent.

In preferred embodiments, fluid flow connectors and their optional features and components are designed to minimize impaction of aerosol deposits along the path between the aerosol generator the patient. For example, and with reference to FIG. 21, at least some portion of the aerosol flowing through connector 200 is believed to follow a main (i.e., substantially direct) aerosol flow path MAFP from aerosol inlet 204 to delivery outlet 206. Portions of the aerosol likely flow along pathways that are outside of the main flow path MAFP—this is illustrated with the additional exemplary aerosol flow path arrows included in FIG. 21. Since sharp turns in an aerosol flow path can induce impaction, it is preferred that main flow path MAFP have an angle a that is less than 90 degrees. Angles of 90 degrees are typical when using a T-connection. Angle α is measured between a reference line (parallel to the aerosol flow as it enters connector 200) and a line defined between a central axis point of the aerosol inlet where the aerosol inlet 204 meets chamber 202 and a central axis point of the delivery outlet where the delivery outlet 206 meets chamber 202. Angle α is preferably less than about 75 degrees, and more preferably less than about 60 degrees.

Even in the absence of sharp turns in the various aerosol conduits, impaction of aerosolized particles may still occur prior to delivery, resulting in deposits that can impair effective delivery of the active agent to the patient. Fluid flow connectors in accordance with the present invention can be adapted for connection to nasal prongs, both for adults and for infants. When delivering an aerosolized active agent through nasal prongs (other delivery devices can be employed), the nasal prongs themselves, due to their relatively small inner diameter, can become a problem area for deposit buildup.

Preferred fluid flow connectors are designed to facilitate the capture of deposits “upstream” of the nasal prongs in an effort to reduce the incidence of deposit build up in the nasal prongs and/or increase the amount of administration time prior to significant deposit buildup. Turning attention again to FIG. 21, a main aerosol flow path MAFP is shown wherein at least a significant portion of the aerosol enters connector chamber 202 via aerosol inlet 204 and then turns toward delivery outlet 206. Without being limited to any one theory, it is believed that relatively large aerosol particles can become separated from the main aerosol flow path, continue along a substantially straight line, and then impact on an opposing chamber 202 surface. In preferred embodiments, the impacting surface can be configured to trap the deposits. By way of example only, chamber 202 may have an internal surface 208 that includes a concave portion 210. The geometry of internal surface 208 helps to define a liquid trap 209 for accepting deposits that become separated from the aerosol flowing through chamber 202, as well as for collecting deposits that were created elsewhere in the system and that are carried to the connector 200 with the aerosol.

An area for collecting deposits within fluid flow connectors, such as, for example, concave portion 210, is preferable located at least partially outside of the main aerosol flow path, so that the collected deposits do not disrupt the active agent delivery to a patient. One manner of accomplishing this is by spacing the deposit collection area (or a portion thereof) away from the delivery outlet 206. Connector embodiments of the present invention are designed and configured to preferably collect deposits in specified areas; however, a person of ordinary skill in the art would readily appreciate that deposits can occur on any and all surfaces of the connectors.

In a further attempt to minimize disruption of delivering the active agent to a patient, fluid flow connector embodiments can employ various means for keeping the collected deposits separated from the aerosol main flow path. One means includes a concavity formed in a wall of the connector chamber—see, e.g., concave portion 210 formed in chamber 202. Another means includes a lip disposed proximate the connector delivery outlet—see, e.g., lip 211. Although connector 200 is shown having both a concavity and a lip, alternative embodiments may incorporate only one or the other.

If there is a significant buildup of deposits (not limited to any specific amount), chamber 202 can be discarded and replaced with a new chamber. Alternatively, deposits can be removed from chamber 202 with a syringe or other suitable device via aerosol inlet 204 or other suitable port (that is preferably sealed). Alternatively, as discussed below, the chamber can include a disposable or removable inserts in which deposits become lodged. Inserts containing lodged deposits may be removed and replaced with fresh inserts. Deposits can be retrieved from chamber 202 while administering an aerosolized active agent to a patient, or alternately, during a non-delivery time period between multiple doses of the active agent.

In view of the above discussion, in certain preferred embodiments of the present invention, it is possible to control the location of deposit collection, isolate the collected deposits from a main aerosol flow path so as to minimize disruption of active agent delivery, and collect deposits for disposal or continued or subsequent active agent delivery.

Deposits that are retrieved from fluid flow connectors of the present invention may be reaerosolized for delivery to a patient. For example, the deposits can be manually retrieved and placed into an aerosol generator. The deposits could also automatically be routed back to an aerosol generator reservoir that is placed substantially below a fluid flow connector. Here, the aerosol is communicated upwardly and into the connector, wherein any deposits could be fed automatically back down to the aerosol generator reservoir via connector features (e.g., a sloped bottom surface), a deposit exit port and flexible tubing or other fluid communication device.

Some of the embodiments of the present invention contemplate delivering an aerosolized active agent to a patient while simultaneously administering other forms of noninvasive respiratory therapy. In preferred embodiments, the respiratory therapy is CPAP (including nCPAP) as discussed in detail herein. To this end, chamber 202 is shown having optional ports 212 and 214 that respectively serve as a ventilation gas inlet and a ventilation gas outlet. In embodiments where CPAP is incorporated, it can be desirable to minimize and/or delay the intermixing of the CPAP gas with the aerosolized active agent. One method of accomplishing this is to include a baffle or flow diverter between the distal end of the aerosol inlet (i.e., the interface between the aerosol inlet and the interior of the chamber) and the ventilation gas (CPAP) inlet. See, for example, FIG. 22, wherein a baffle 207 is included that generally directs the flow of the ventilation gas, at least initially, along a fluid flow pathway labeled VPW. The aerosol generally follows a fluid flow pathway labeled APW. The two fluid flow pathways merge in an area proximate the delivery outlet 206.

Two other optional ports 216 and 218 are shown extending from chamber 202. Port 216 can be utilized for proximal pressure measurements associated with the administration of CPAP. Port 218 can be used for removing deposits that are trapped in chamber 202 without having to remove devices inserted into aerosol inlet 204. For this application, port 218 can employ a septum that can be penetrated with a standard needle and syringe.

One of ordinary skill in the art would readily appreciate that the number, arrangement, size, and geometry of the features associated with chamber 202, including those described above, can vary considerably without departing from its useful function and the scope of the claims appended hereto.

In other embodiments, the aerosolized active agent is not delivered in conjunction with CPAP. In still other embodiments, the aerosolized active agent is delivered without simultaneous delivery of other forms of noninvasive respiratory therapy.

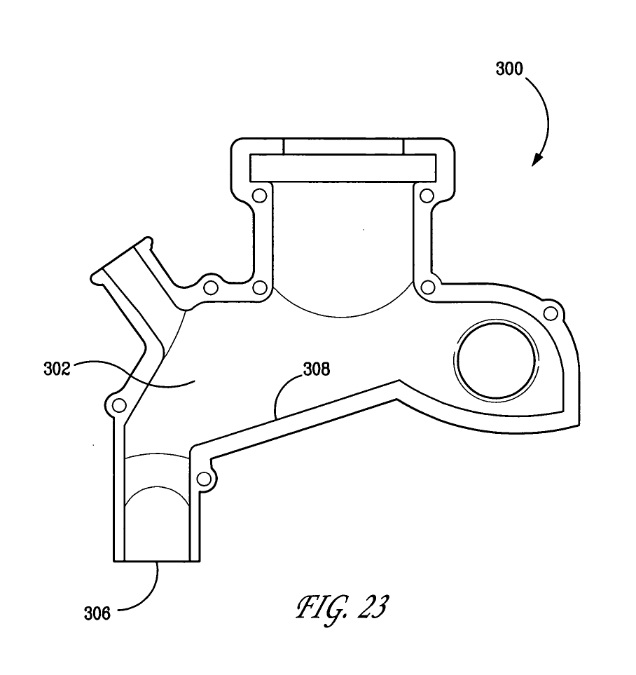

Rather than discarding a fluid flow connector containing deposits, or removing the deposits to permit additional usage of the connector, the chamber can include one or more features that facilitate communication of impacted deposits to the patient. That is, both the aerosolized active agent and the deposits can be delivered to the patient to maximize the delivery efficiency of the active agent. For example and with reference to FIG. 23, another exemplary fluid flow connector 300 is shown that includes a chamber 302 having an internal surface 308 that is downwardly angled in a direction towards delivery outlet 306. Deposits that impact internal surface 308 can essentially slide down to delivery outlet 306 with the aid of gravity, and optionally a wetting agent applied to internal surface 308. Pressure associated with the flowing aerosol, and CPAP ventilation gas if incorporated, will also tend to “push” deposits down angled surface 308.

Each of connectors 200 and 300 are configured and shown for receiving an aerosolized active agent from above the connector—that is, through an aerosol inlet disposed in an upper wall. However an aerosol generator can be disposed below or beside the fluid flow connector, such that an aerosol inlet accordingly is positioned in a sidewall or bottom wall of the connector. In these embodiments, one or more internal surfaces, including or other than a bottom surface, can serve as an impact surface that is configured for either trapping deposits associated with an aerosolized active agent, or for communicating the deposits to the delivery outlet so that both the aerosolized active agent and the deposits are delivered to the patient. One potential advantage to having an aerosol generator below the fluid flow connector, so as to effectively “shoot” the aerosol in an upward direction, is that gravity may slow the aerosol down to reduce impaction and the resulting buildup of deposits on internal chamber surfaces. As noted above, where an aerosol generator is placed below a fluid flow connector, any deposits initially collected in the connector can optionally be routed back to the aerosol generator for re-aerosolization.

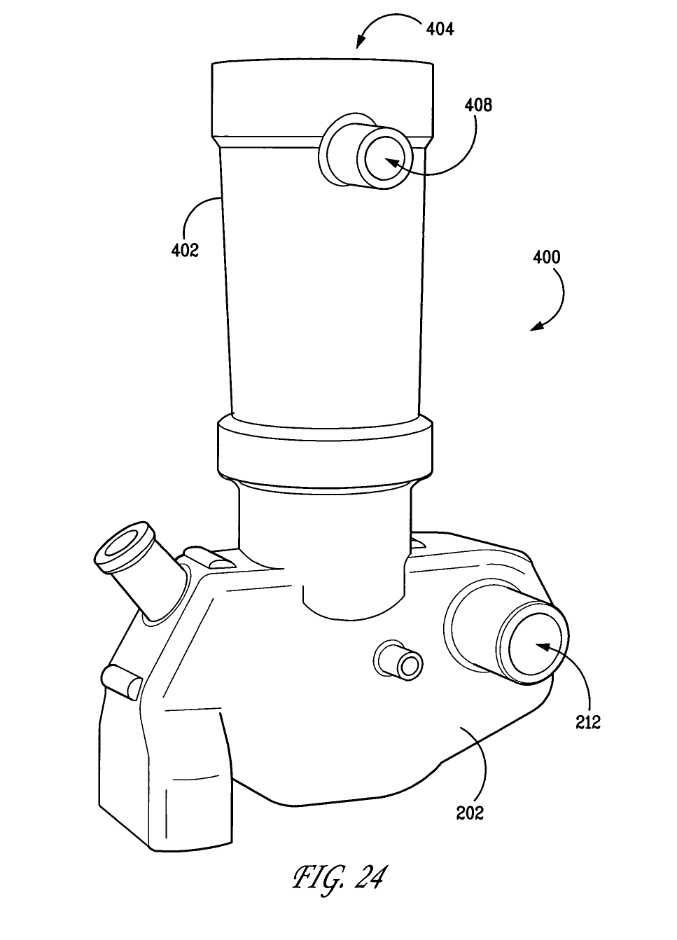

Referring now to FIGS. 24-25, an alternative fluid flow connector 400 is shown including chamber 202 (as shown and described with reference to FIGS. 18-21) and an aerosol conditioning vessel 402 inserted into aerosol inlet 204. It should be understood that the reason fluid flow connectors 200 and 300 are shown in the absence of an aerosol conditioning vessel is because the conditioning vessel is an optional feature that should not be read into claims that do not specifically recite the same.

Aerosol conditioning vessel 402 has an inlet 404 for receiving an aerosolized active agent, an outlet 406 that is in fluid communication with aerosol inlet 204, and conditioning gas inlets 408. Conditioning gas can be supplied from an independent source, or can alternatively be “split off of” CPAP ventilation gas that is also being introduced into chamber 202 via inlet 212. Where a portion of the ventilation gas is being supplied to the conditioning vessel, tubing may be employed that stems from the ventilation tubing and is connected to inlet 408, or a conduit or channel (located internally or externally) can be employed by connector 200 that extends from chamber 202 to the conditioning vessel to communicate some of the ventilation gas to the conditioning vessel.

Conditioning vessel 402 preferably has two diametrically opposed gas inlets 408, but the vessel may employ only one gas inlet, or more than two. When there are two or more gas inlets, it is preferred to dispose them symmetrically about the circumference of the conditioning vessel (“radially symmetric”) to facilitate substantially uniform gas flow into the conditioning vessel-non-uniform gas flow may cause deposits to form on the sidewalls of the conditioning vessel. It should be noted however, that asymmetric designs are still within the scope of the present invention, and clinicians may desire non-uniform gas flow in certain applications. Conditioning vessel embodiments that employ only one gas inlet can be designed to maintain radial symmetry of the conditioning gas flow. For instance, the conditioning gas inlet can be placed behind the aerosol generator, with the conditioning gas flow directed in the same direction as the aerosol. In this embodiment, the conditioning gas passes around the aerosol generator and then meets and envelopes the aerosol stream again, with both the conditioning gas and the aerosol moving in the same direction. Radial symmetry would be maintained such that the conditioning gas would not be blowing the aerosol against a wall. Alternatively, the conditioning vessel may include internal features (e.g., a mesh or set of slits, acting as a diffuser), to ensure radial symmetry of the sheath gas flow once the gas is inside the vessel, prior to communication with the aerosol.

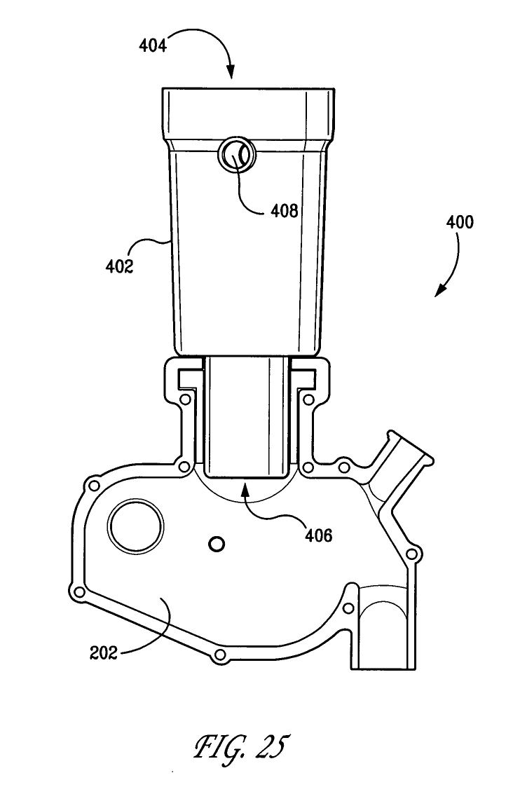

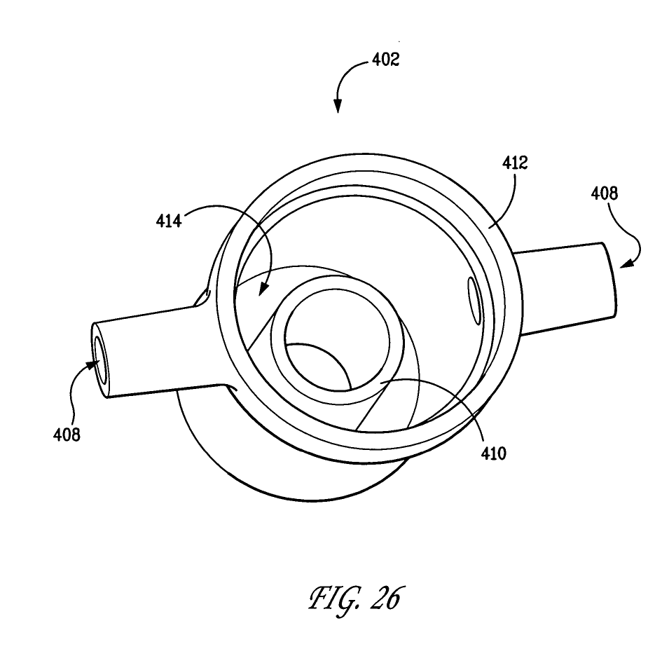

As shown in FIG. 26, aerosol conditioning vessel 402 is basically two cylindrical bodies connected or formed together. Cylindrical body 410 extends partially within cylindrical body 412 to define an annular liquid trap 414 for collecting deposits associated with an aerosolized active agent flowing through the conditioning vessel. Aerosol conditioning vessel 402 may employ a port (not shown) for retrieving deposits collected in liquid trap 414.

Referring again to FIG. 24, conditioning vessel 402 is a separately manufactured component and is designed to be removably inserted into aerosol inlet 204, preferably through a cross slit valve, although other types of seals, gaskets, etc. can be used to prevent appreciable leakage of the aerosolized active agent. In some embodiments, the conditioning vessel is simply held in engagement with chamber 202 by friction and dimensional constraints. During operation, however, the aerosol can lubricate component surfaces, and thereby reduce the frictional fit to a point where the conditioning vessel becomes disengaged from chamber 202. To prevent premature disengagement, locking features (not shown) can be included on each of the components. For example, the components can have mating screw threads on respective engaging surfaces, so that the conditioning vessel can be inserted and then rotated to effect a secure engagement. In one preferred embodiment, aerosol inlet 204 has an L-shaped groove and the conditioning vessel has a post that can fit into the groove, whereby the conditioning vessel is inserted axially and then rotated (e.g., by a quarter turn) to lock the components in place.

In alternative embodiments, at least a portion of the conditioning vessel and the chamber are formed together (e.g., via injection molding). This one-piece design may employ one or more liquid traps for collecting deposits associated with the aerosolized active agent, and one or more ports for retrieving the deposits. In other alternative embodiments, the aerosol generator, fluid flow connector, and optionally conditioning vessel, are formed together as a one-piece design. These components can also be manufactured separately and then permanently affixed to each other.

A conditioning vessel can be employed to alter the flow of the aerosolized active agent, alter the characteristics of the aerosol, or both. Conditioning gas can help direct the flow of the aerosol through fluid flow connectors of the present invention—i.e., improving the direction coherence of the stream of aerosol particles. Conditioning gas can, in some embodiments, alter the characteristics of the incoming aerosol by modifying the ratio of active agent to medium, or by reducing the mass median aerodynamic diameter of the aerosol particles, for example.

Active agent concentrating chambers may be utilized with fluid flow connectors of the present invention. These concentrating chambers would typically be disposed between the aerosol generator and the main chambers (e.g., 202 and 302) of the connectors as discussed above. For example, an exemplary concentrating chamber 500 is shown in FIG. 27 disposed above a fluid flow connector 510. Preferred concentrating chambers are intended to facilitate the creation of a high density aerosol cloud that can then be communicated to a patient for maximizing the delivery rate of the active agent. One way of generating a high density aerosol cloud is by restricting the flow of the aerosol from the aerosol generator to a delivery chamber associated with a fluid flow connector, so that the active agent is concentrated prior to delivery. For example a simple flexible tube (or other chamber) containing a one-way valve can be placed between the aerosol generator and the delivery chamber. The one-way valve (see, e.g., valve 520 in FIG. 27) will normally be closed, and negative pressure generated by a patient's inhalation will actuate the valve and permit a concentrated portion of the aerosolized active agent to be delivered. Restricting the aerosol can be accomplished by any number of techniques other than incorporating a one-way valve between the aerosol generator and the delivery chamber.

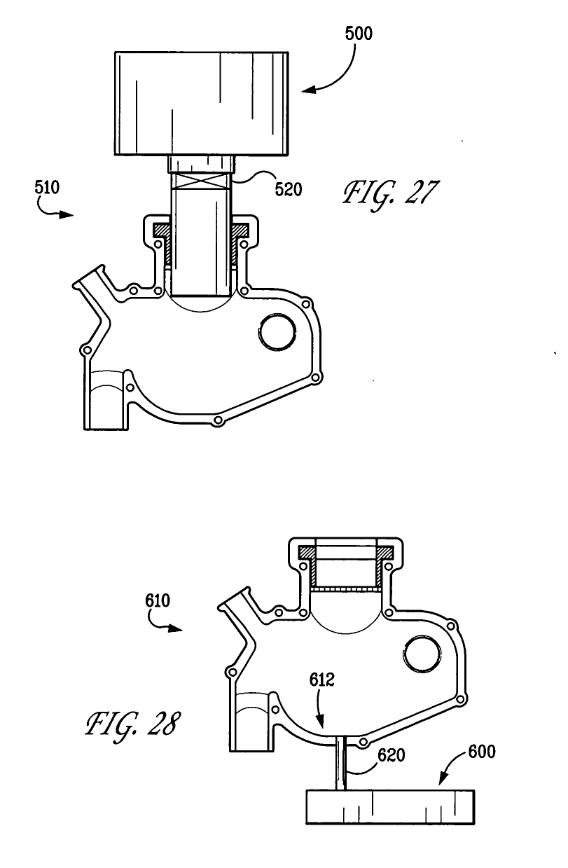

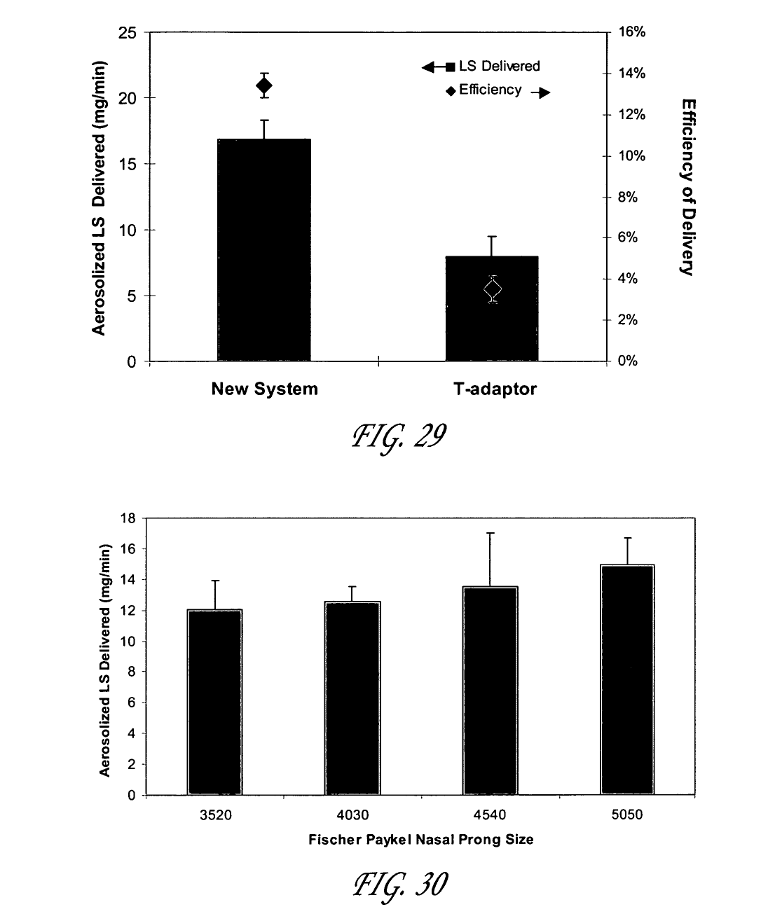

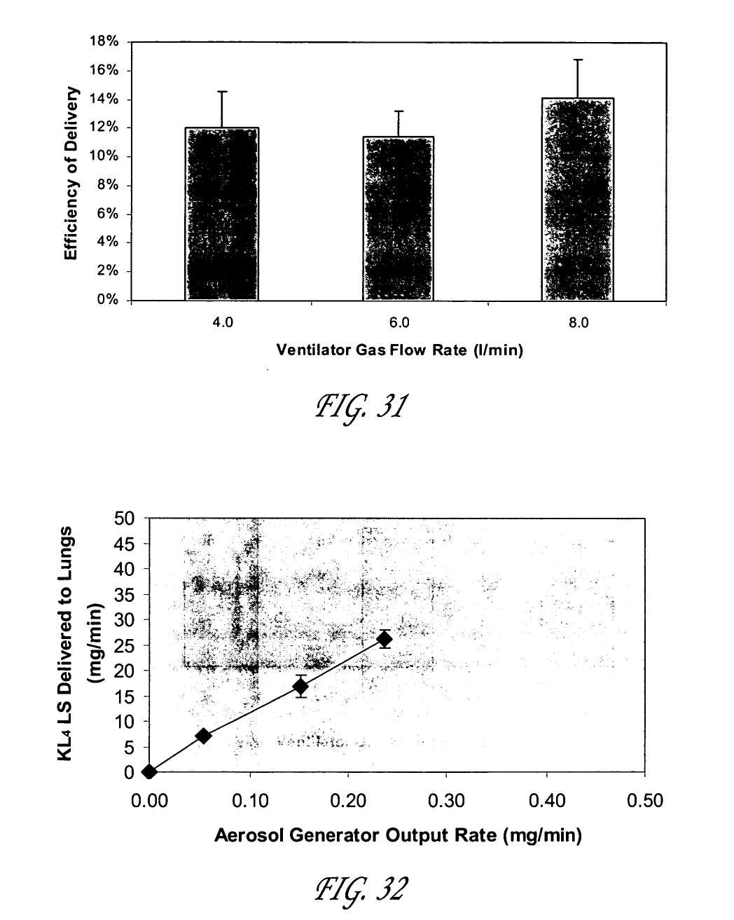

Fluid flow connectors of the present invention can employ a collection reservoir that is disposed below the delivery chambers for sequestering deposits associated with an aerosolized active agent. The collection reservoirs provide for an “automatic” removal of deposits from a fluid flow connector's chamber as compared to manual removal with a syringe or other suitable device. The collection reservoirs can be employed as additional means to collecting deposits (e.g., traps, chamber internal geometry), or may serve as an alternative to the aforementioned deposit collecting features. The collection reservoirs can be connected either directly or indirectly (e.g. with a conduit) to the delivery chambers. In some forms, the collection reservoirs are disposable, such that a filled (partially or completely) collection reservoir can be removed and a new one connected for accepting subsequent deposits. The collection reservoirs can be configured to accept disposable inserts, such as, for example, absorbent nonwoven pads. They can also include a port for retrieving deposits and/or for venting pressure. Referring to FIG. 28, an exemplary collection reservoir 600 is shown. Collection reservoir 600 is connected to a fluid flow connector 610 via a conduit 620. Since fluid flow connector 610 includes a concavity 612, deposits can initially be collected in the concavity and then drain into collection reservoir 600. This “draining” effect provides yet another means for keeping collected deposits separated from the aerosol main flow path.

Although the figures and description focus on embodiments wherein the aerosol generator, fluid flow connectors and optional conditioning vessels are positioned close to a patient, alternative component locations are contemplated by the present invention. For example, an aerosol generator and fluid flow connector (examples of which are shown and described above) can be located distal from a patient, with the aerosolized active agent communicated to the patient via flexible tubing, an optional second connector (which may or may not be designed to trap deposits), and an appropriate interface, such as, for example, nasal prongs.

The methods and systems described herein are particularly useful in rescue and prophylactic treatment of infants with RDS and in adults with ARDS. The actual dosage of active agents will of course vary according to factors such as the extent of exposure and particular status of the subject (e.g., the subject's age, size, fitness, extent of symptoms, susceptibility factors, etc). By “effective dose” herein is meant a dose that produces effects for which it is administered. The exact dose will be ascertainable by one skilled in the art using known techniques. In one exemplary embodiment, the effective dose of lung surfactant for delivery to a patient by the present methods will be from about 2 mg/kg surfactant TPL to about 175 mg/kg surfactant TPL. The length of treatment time will also be ascertainable by one skilled in the art and will typically depend on dose administered and delivery rate of the active agent. For example, in embodiments wherein the delivery rate of aerosol to a patient is about 0.6 mg/min, greater than 100 mg of aerosol can be delivered in less than a 3 hour time frame. It will be understood by the skilled practitioner that a lower delivery rate will correspond to longer administration times and a higher delivery rate will correspond to shorter times. Similarly, a change in dose will effect treatment time.

In addition, the methods and systems are also useful in treating other clinical disorders as seen in infants and other pediatric patient populations such as, by way of example cystic fibrosis, intervention for infectious processes, bronchiolitis, and the like.

It is contemplated that patients that could benefit from the methods and systems described herein ranges from premature infants born at about 24 weeks gestation to adults. As infants mature they transition from nasal to oral breathers and as such it is contemplated that the nature of the delivery system would be modified for use via oral delivery systems including face masks and the like.

It is further contemplated that adult patients who suffer from obstructive sleep apnea and upper airway resistance syndrome and other disorders that are remedied at least in part by CPAP. As such, those adults will also benefit.

Patients inflicted with other respiratory disorders can benefit from the methods and systems of the invention. These respiratory disorders include, for example, but are not limited to the disorders of neonatal pulmonary hypertension, neonatal bronchopulmonary dysplasia, chronic obstructive pulmonary disease, acute and chronic bronchitis, emphysema, bronchiolitis, bronchiectasis, radiation pneumonitis, hypersensitivity pneumonitis, acute inflammatory asthma, acute smoke inhalation, thermal lung injury, asthma, e.g., allergic asthma and iatrogenic asthma, silicosis, airway obstruction, cystic fibrosis, alveolar proteinosis, Alpha-1-protease deficiency, pulmonary inflammatory disorders, pneumonia, acute respiratory distress syndrome, acute lung injury, idiopathic respiratory distress syndrome, idiopathic pulmonary fibrosis, sinusitis, rhinitis, tracheitis, otitis, and the like. Accordingly, the present invention provides methods, systems, and devices for treating these diseases in a patient.

### EXAMPLES

Unless otherwise stated all temperatures are in degrees Celsius. Also, in these examples and elsewhere, abbreviations have the following meanings:
bpm=breaths per minute
cm=centimeter
DPPC=dipalmitoyl phosphatidylcholine
l/min=liters/minute
mg=milligram
min=minute
ml=milliliter
mM=millimolar
mm=millimeter
PA=palmitic acid
POPG=palmitoyloleoyl phosphatidylglycerol
rpm=revolutions per minute
μl=microliter
μm=micrometer

### Example 1

Preparation of Exemplary Lung Surfactant Comprising KL4

The basis of the composition is a combination of DPPC, POPG, palmitic acid (PA) and a 21 mer peptide, sinapultide (KL4) consisting of lysine-leucine (4) repeats. The peptide was produced by conventional solid phase t-Boc chemistry and has a molecular weight of 2469.34 units as the free base. The components were combined as described below, in the mass ratio of 7.5:2.5:1.5:0.267 as DPPC:POPG:PA:KL4 to produce a stable colloidal dispersion in an aqueous trimethamine (20 mM) and sodium chloride (130 mM) buffer adjusted to a pH of 7.6 at room temperature. Concentrations of 10, 20, and 30 mg/ml of phospholipid content were produced.

Accurately weighed powders of DPPC, POPG, PA, and KL4 were sequentially added to an appropriately sized round bottom flask containing sufficient heated ethanol at 45° C. to dissolve the components. The ethanol is present in excess of 120:1 (volume:mass). Each active was added in conjunction with a 5-minute burst of ultrasonication within a water bath. After all of the actives have been added a further 5-minute burst of ultrasonication is applied. The ethanolic solution is then rotary evaporated (temperature 50-55° C., rotary speed 50 rpm and vacuum of 0 mbar) to produce a persistent thin film on the bottom of the flask. Residual ethanol was then removed by storing the flask for at least 12 hours within a vacuum desiccator.

The dried film was hydrated in tris-acetate and then salt was added post hydration at a temperature of 50-55° C. in combination with waterbath sonication for approximately 30 minutes ensuring complete hydration of the film and the absence of visible aggregates in the final aqueous dispersion.

Reverse phase high performance liquid chromatographic (HPLC) analysis was used to establish the integrity and recovery of the phospholipids (DPPC, POPG) and free fatty acids (PA) used in the preparation above. Analysis was performed on a chromatographic work-station (HP1100, Agilent Technologies, Palo Alto, Calif.). A Zorbax-C18 column (5μ, 250×4.6 mm) was employed to separate and resolve the formulation components using a mobile phase consisting of 90% Methanol, 6% acetonitrile, 4% water and 0.2% trifluoroacetic acid by volume, running at 1 ml/min. Column temperature was maintained at 60° C. The injection volume was 20 μl. An evaporative light scattering detector was used for detection of the compounds.

Aliquots of the dispersion were subsequently transferred to borosilicate vials and stored at 2-8° C.

### Example 2

Comparison of Conditioned Aerosol with Unconditioned Aerosol

A composition of Example 1 was prepared at a concentration of 15 mg/ml. FIG. 12 illustrates in schematic view the system that was employed. It should be noted that there is an outlet in-line with line 70 that is not shown. Specifically, an Aeroneb Pro nebulizer (Aerogen, Inc., Mountain View, Calif.), was used to aerosolize the composition. The aerosol was conditioned by the system and the conditioned aerosol was directed toward nasal prongs (Fisher-Paykel, NZ). A ventilator was used to create a CPAP-producing gas flow and was set at 6 l/min flow rate and 5 cm H2O CPAP. The infant breathing pattern was mimicked using a ventilator that was set at 54 bpm and tidal volume of 6.4 ml. The ventilator was connected downstream of the collection system (not shown). Without the sheath gas, negligible aerosol passed through the nasal prongs and most of the aerosol deposited on the system components. When the conditioning gas flow rate was set at 1 l/min and at room temperature, an average of 0.64 mg/min of the conditioned aerosol was collected over a ten-minute-run period (n=2).

The results are presented in FIG. 13 which illustrates the rate of conditioned aerosol collected in an unconditioned system and an exemplary conditioned system.

### Example 3

Effect of Conditioning Gas Flow Rate and Temperature on the Aerosol Amount Emerging Through the Nasal Prongs

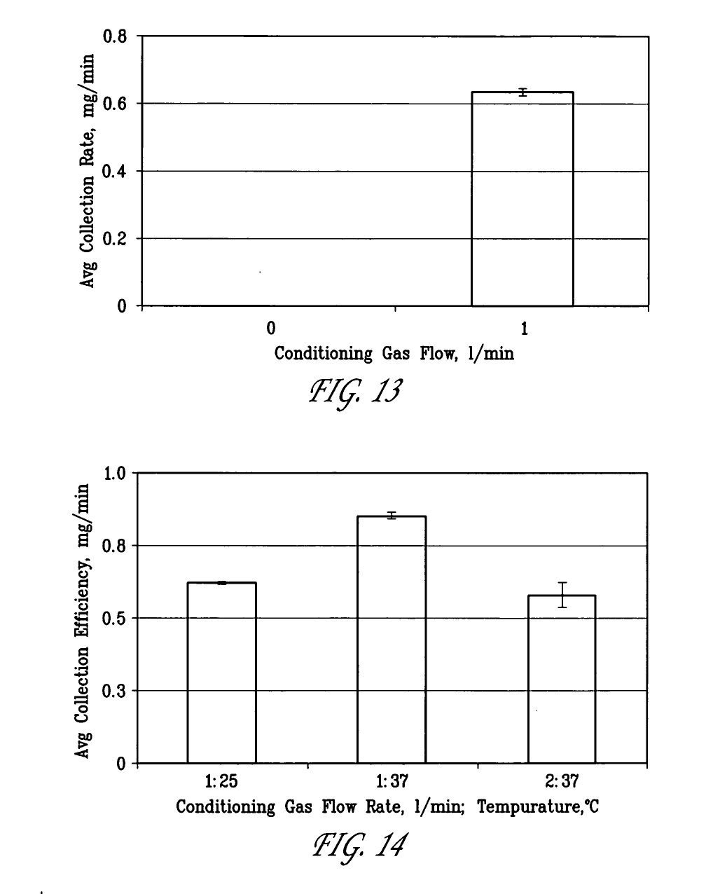

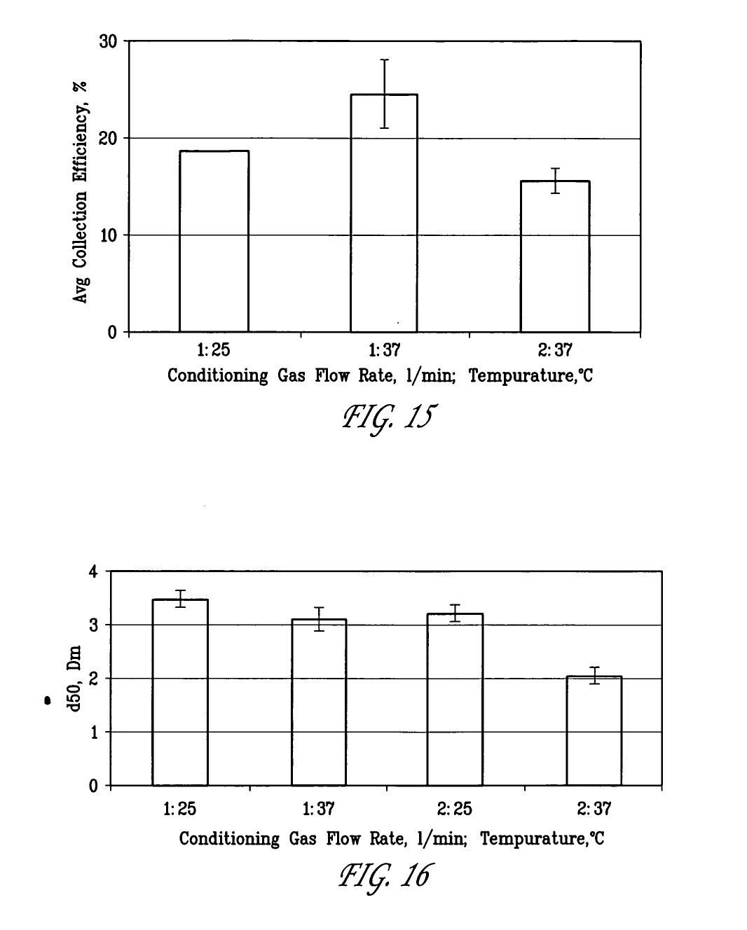

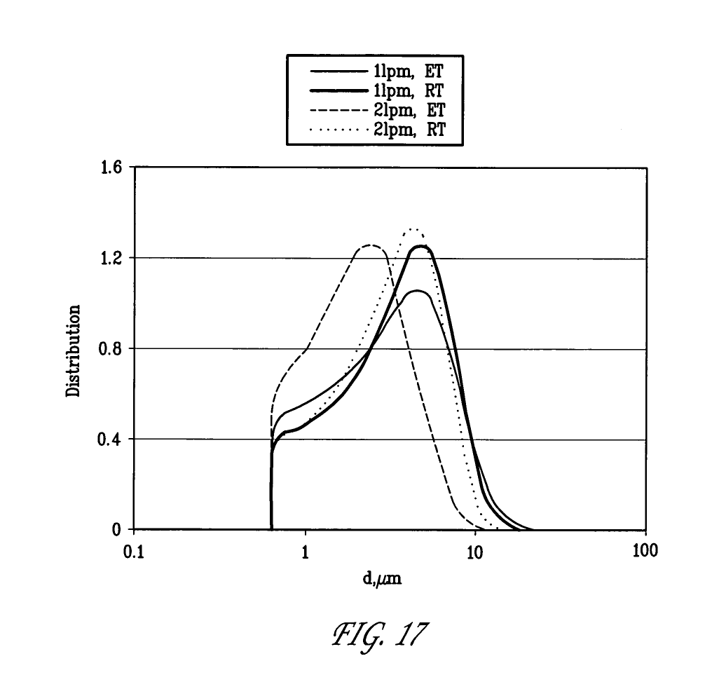

The same setup and experimental conditions as used in Example 2 were employed to examine the effect of conditioning gas flow rate and temperature on the amount of aerosol emerging from the delivery apparatus. In this example, nasal prongs were employed. With a conditioning gas flow rate of 1 l/min, increasing the gas temperature from 25 to 37° C., increased the amount of conditioned aerosol emerging through the prongs (collected in the filter) by about 38%. The results are presented in FIG. 14. In this example, higher conditioning gas temperature provides more energy to evaporate moisture in the droplets creating smaller droplets, and thus decreased deposition losses by particle coalescence and/or deposition on surfaces. At the same gas temperature (37° C.), increasing the conditioning gas flow rate from 1 l/min to 2 l/min decreased the amount of aerosol collected in the filter by about 33%, due to higher aerosol dilution with higher gas flow rate. FIG. 15 shows the percentage of conditioned aerosol that passed through the prong with different conditioning gas flow rates and temperatures, i.e. 19% for 1 l/min at 25° C., 25% for 1 l/min at 37° C. and 16% for 2 l/min and 37° C.

### Example 4

Effect of Conditioning Gas Flow Rate and Temperature on the Aerosol Size Emerging Through the Nasal Prongs

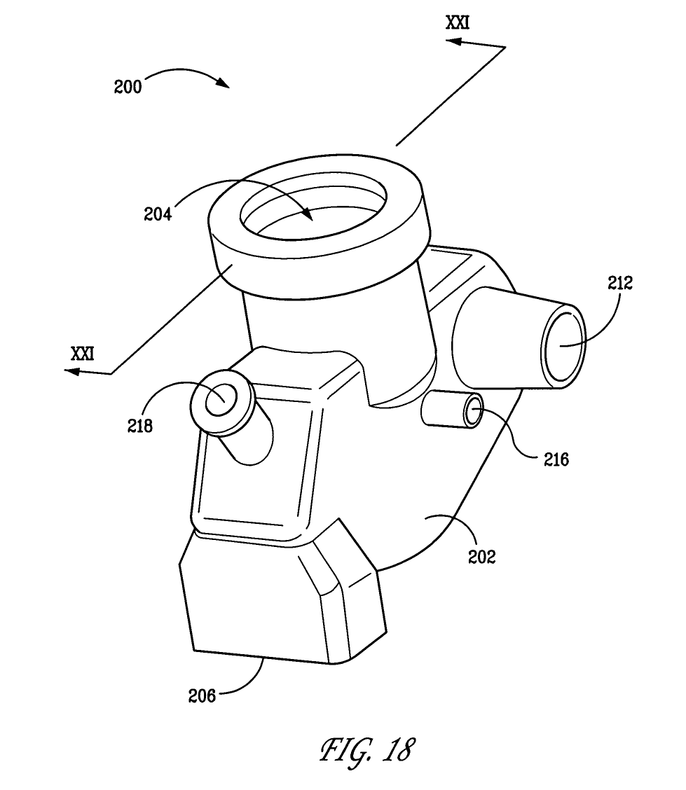

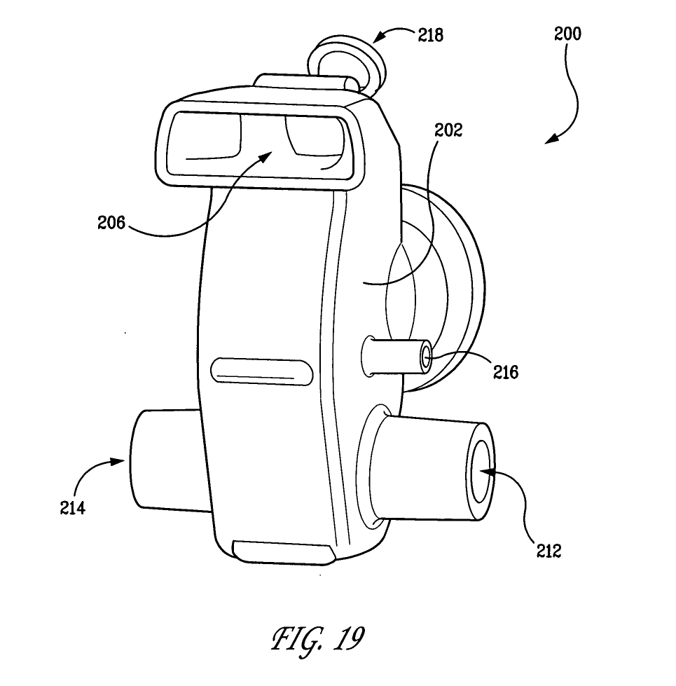

The same experimental setup and conditions as used in Example 2 were employed. The conditioned aerosol size and size distribution were determined using laser diffraction analysis (Sympatec Helos/BF, Sympatec, Princeton, N.J.). As indicated in FIGS. 16 and 17, increasing the conditioning gas temperature from 25 to 37° C., decreased aerosol volume median diameter (d50), i.e. 3.5 to 3.1 μm for 1 l/min and 3.17 to 2.0 μm using the 2 l/min sheath gas flow rate. The effect of conditioning gas temperature on aerosol size is more pronounced at a higher gas flow rate.

In FIG. 17, “lpm” refers to liters per minute, “ET” refers to elevated temperature or 37° C., and “RT” refers to room temperature or 25° C.

### Example 5

Effect of Lung Deposition of Aerosolized KL4 Lung Surfactant in Healthy Adults

A study of healthy adult humans was performed using an exemplary device of the present invention. The fraction of aerosolized KL4 lung surfactant deposited in the lungs was measured. Table 1 summarizes this data and shows that 16 to 25% of the aerosolized drug was deposited in the lungs in healthy adult humans.

TABLE 1
Fractional lung deposition of aerosolized
KL4 Lung Surfactant in healthy adults

| Volunteer Number | % DD to lung |
|---|---|
| 1 | 16.1 |
| 2 | 21.8 |
| 3 | 20.7 |
| 4 | 25.3 |
| 5 | 22.2 |
| 6 | 25.3 |
| Mean | 21.9 |

### Example 6

Surfaxin® Aerosol CPAP Trial

Four subjects, three Hispanic females and on Caucasian male with a mean gestational age of 30.7 weeks, birth weight range 1095-1744 grams were treated with Surfaxin® aerosol using an exemplary device of the present invention. Apgar scores ranged from 7-9 at one minute to 8-9 at five minutes. Surfaxin® aerosol treatment time ranged from 3 hours 19 minutes to 4 hours 22 minutes. The FiO₂ for Subject 1 at baseline was 0.40. After one treatment with Surfaxin® aerosol the FiO₂ for Subject 1 was reduced to 0.21. The FiO₂ for Subject 2 at baseline was 0.60. After one treatment with Surfaxin® aerosol the FiO₂ for Subject 2 was reduced to 0.24. Similarly, FiO₂ for Subjects 3 and 4 were 0.28 and 0.40 at baseline, respectively. Although Subject 3 had two treatments with Surfaxin® aerosol, similar reductions in FiO₂ were seen with a reduction in FiO₂ to 0.22 and 0.23, respectively. The following exemplary protocol was followed:

1. Inserted one vial of Surfaxin® into a warming cradle

2. Warmed for about 15 to 20 minutes

3. Drew 6 mL into a 10 mL syringe to achieve a 20 mg/mL concentration

4. Drew 3 mL preservative free saline into the syringe

5. Drew 1 mL air into the syringe

6. Gently swirled the syringe to mix the Surfaxin® with the saline.

7. Placed Support Fixture on bassinette. Padded well.

8. Attached appropriate sized nasal prongs to the outlet port of the Prong Adapter.

9. Connected the CPAP inspiratory line and expiratory line of the ventilator circuit to the large open ports on the Prong Adapter.

10. Pulled out male fitting from pressure sensor line and cut tubing ¼″ to ½″. Connected the CPAP pressure sensor tubing to the smallest port (proximal pressure port) on the Prong Adapter. Ensured a snug fit.

11. Positioned the Prong Adapter over the infant with the nasal prongs positioned properly in the infant's nares.

12. Slid inspiratory and expiratory lines of the ventilator tubing through the channels on the Support Fixture: adjusted the height of the holster on each side of the Support Fixture to the desired level. Inserted the ventilator tubing (inspiratory and expiratory line) into the appropriate holster. Snapped ventilator tubing into Support Fixture. Ensured nasal prongs remained in the infant's nares.

13. Attached a Pall Filter to the expiratory line of the CPAP circuit.

14. Attached distal end of inspiratory line to the Fisher Paykel humidifier. Connected heating wires of the inspiratory and expiratory lines into appropriate connections on the humidifier.

15. Inserted proximal and distal temperature probes to ventilator circuit.

16. Placed an appropriate sized nasogastric tube, which corresponds to the infant's birth weight, open to air into the infant's stomach.

17. Initiated CPAP ventilation and adjust to appropriate flow rate for an operating pressure of 5-6 cm H₂O.

18. Transported to NICU.

19. Connected the Aeroneb Pro Control Module Cable to the Aeroneb Pro nebulizer head. Ensured opposite end of the Control Module Cable is connected to the Aeroneb Pro Control Module.

20. Confirmed Aeroneb Pro Control Module was plugged into a standard 110v electrical outlet and was operational.

21. Connected the two ¼″ ID tubes (8″ lengths) from the Y-connector to the two ports on the sides of the Conditioning System. Connected the remaining ¼″ ID tube (6′ length) from the Y-connector to the barbed end of the adaptor connected to the FloTec flow meter attached to the blended gas outlet of the Infant Star ventilator. At this point, no airflow should be started.

22. Turned on the FloTec flow meter (at back of ventilator) attached to the blended gas outlet to 1 liter/minute by turning the black dial until a ‘1’ is shown in the display.

23. Removed orange protective cap from the Aeroneb Pro nebulizer head. Attached the Aeroneb Pro nebulizer head directly to the entry port of the Conditioning System.

24. Attached the Conditioning System together with the nebulizer head by inserting the outlet port of the Conditioning System through the slit valve port of the Prong Adapter. Supported the bottom of the Prong Adapter while inserting the Conditioning System into the Prong Adapter. Ensured nasal prongs remained in the infant's nares.

25. Removed 16-gauge needle from the 10 mL syringe in which the Surfaxin® was diluted. Added the diluted 9 mLs Surfaxin® 20 mg/mL through the leur-tip of the syringe, into the reservoir of the nebulizer head.

26. Recorded the amount of Surfaxin® added to the nebulizer in the Case Report Form.

27. The Surfaxin® Drug Delivery System was then ready for operation.

28. Confirmed that the sheath gas airflow meter is set to 1 liter/minute and adjust if not set correctly. Ensured CPAP pressure is maintained.

29. Turned on the Aeroneb Pro Control Module by pressing and holding the “blue button” for ˜3 seconds. The indicator light next to the “30 min” mark on the module became illuminated. The control module must be re-started every 30 minutes.

30. Began aerosolization. Watched for aerosol being generated through the Surfaxin® Delivery System.

31. Inserted a vial of Surfaxin® into the heating block.

32. Suctioned the baby's mouth as necessary but at least every 30 minutes.

33. Turned the nebulizer off at the Aeroneb Pro Control Module (blue button, press once and release) and removed the Conditioning System with the nebulizer head from the Prong Adapter through the cross-slit valve: pulled straight up while ensuring the prong adapter did not move. If resistance was met, gently rotated the device left and right while continuing to remove it. Set the Conditioning System and nebulizer aside.

34. Inserted a disposable, sterile 3 ml syringe (without a needle) through the cross-slit valve at the top of the Prong Adapter and removed the accumulated material from the drip trap. The valve should close tightly enough around the syringe to ensure that CPAP is not interrupted (some airflow may be felt passing through the valve, this is normal and should not affect the CPAP).

35. Gently removed the Aeroneb Pro Control Module Cable from the nebulizer head.

36. Gently removed the nebulizer head from the top of the Conditioning System.

37. Switched the sheath gas tubing from the used Conditioning System to a new Conditioning System. Discarded the used Conditioning System in appropriate medical waste receptacle.

38. Replaced Pall filter. When ready quickly detach the expiratory line from the old filter, remove it and reconnect expiratory line to the new filter. When the new filter is in place the CPAP will re-adjust to the original set point over the course of a few minutes.

39. Rinsed the underside of the nebulizer with sterile water.

40. Gently reattached the Aeroneb Pro nebulizer head to the new Conditioning System.

41. Connected the Aeroneb Pro Control Module Cable to the Aeroneb Pro nebulizer head.

42. Gently inserted the new Conditioning System together with the nebulizer head through the slit valve port of the Prong Adapter. If any resistance was met, rotated the Conditioning System left and right while inserting. Supported the bottom of the Prong Adapter while inserting the new Conditioning System and nebulizer head.

43. Lifted the filler cap on the nebulizer head. Filled the reservoir of the nebulizer head with Surfaxin® 20 mg/mL. Removed the 16-gauge needle from the 10 mL syringe in which the Surfaxin® was diluted. Added the diluted 9 mLs Surfaxin® 20 mg/mL through the luer-tip of the syringe, through the filler cap into the reservoir of the nebulizer head. Closed filler cap when finished.

44. Turned on the Aeroneb Pro Control Module by pressing the “blue button” for ˜3 seconds. The indicator light next to the “30 min” mark on the module became illuminated. The control module must be re-started every 30 minutes.

45. Turned off the nebulizer at the Aeroneb Pro Control Module (pressed the blue button).

46. Removed the Conditioning System with nebulizer head from the Prong Adapter.

47. Removed the Aeroneb Pro nebulizer head from the Conditioning System.

48. Disconnected Y-tubing (¼″×6″×6′) from ventilator and disposed of per hospital protocol.

49. The CPAP circuit could remain operational with no further changes, however to completely remove the apparatus continued with the steps below:
a. Turned off the Infant Star ventilator.
b. Removed the ventilator tubes from the Device Support Unit and withdrew the nasal prongs from the infant's nares.

50. Unplugged the Aeroneb Pro Control Module Cable from the nebulizer head.

The figures and examples of specific embodiments for carrying out the present invention are offered for illustrative purposes only, and are not intended to limit the scope of the present invention in any way. From the foregoing description, various modifications and changes in the methods, devices, and systems will occur to those skilled in the art. All such modifications coming within the scope of the appended claims are intended to be included therein.

The disclosures of all publications, patents and patent applications cited herein are hereby incorporated by reference in their entirety.

What is claimed:

1. A method for delivering an aerosolized active agent to a patient, comprising the steps of:
obtaining the active agent as a mixture in a medium;
generating a stream of particles of the mixture with an aerosol generator to produce the aerosolized active agent; and
communicating the aerosolized active agent to and through a fluid flow connector that includes an outlet for delivering the aerosolized active agent to the patient, the fluid flow connector configured to direct the aerosolized active agent along a main aerosol flow path to the outlet and to be capable of collecting deposits associated with the aerosolized active agent in an area that is located at least partially outside the main aerosol flow path, thereby delivering the aerosolized active agent to the patient.

2. The method of claim 1, further comprising the step of administering noninvasive pulmonary respiratory therapy to the patient.

3. The method of claim 2, wherein the noninvasive pulmonary respiratory therapy is continuous positive airway pressure (CPAP).

4. The method of claim 2, further comprising the step of:
retrieving the any collected deposits from the fluid flow connector while simultaneously conducting the step of administering noninvasive pulmonary respiratory therapy to the patient.

5. The method of claim 4, wherein the step of communicating the aerosolized active agent to and through a fluid flow connector is stopped while retrieving the any collected deposits from the fluid flow connector.

6. The method of claim 1, wherein the aerosolized active agent comprises a lung surfactant.

7. The method of claim 6, wherein the lung surfactant is an animal-derived or synthetic surfactant.

8. The method of claim 7, wherein the synthetic surfactant comprises a hydrophobic peptide selected from the group consisting of KL4, RL4, RL8, R2L7, RL4CL3, RL5CL3, RL3CL3, polylysine, magainans, defensins, iseganan, histatin, and combinations thereof.

9. The method of claim 8, wherein the hydrophobic peptide is KL4.

10. The method of claim 9 wherein the hydrophobic peptide is suspended in an aqueous dispersion of phospholipids and free fatty acids or fatty alcohols.

11. The method of claim 1, wherein the mixture comprises a wetting agent.

12. The method of claim 1, wherein the medium is saline.

13. The method of claim 1, further comprising the step of:
retrieving the any collected deposits from the fluid flow connector.

14. The method of claim 13 further comprising the steps of:
aerosolizing the deposits to produce a supplemental volume of the aerosolized active agent; and
delivering the supplemental volume of the aerosolized active agent to the patient.

15. A method for delivering a first and second aerosolized active agent to a patient comprising the steps of:
obtaining the active agent as a mixture in a medium;
generating a first stream of particles of the mixture with an aerosol generator to produce a first aerosolized active agent;
communicating the first aerosolized active agent to a fluid flow connector that includes an outlet for delivering the first aerosolized active agent to the patient, the fluid flow connector configured to direct the aerosolized active to the outlet while collecting deposits associated with the aerosolized active agent in or on a part of the fluid flow connector that is substantially spaced apart from the outlet;
delivering the first aerosolized active agent to the patient;
retrieving deposits from the fluid flow connector;
generating a second stream of particles of the mixture with an aerosol generator to produce a second aerosolized active agent; and
delivering the second aerosolized active agent to the patient.

16. A method for delivering an aerosolized active agent to a patient, the method comprising the steps of:
obtaining the active agent as a mixture in a medium;
generating a stream of particles of the mixture with an aerosol generator to produce the aerosolized active agent;
communicating a volume of the aerosolized active agent to a fluid flow connector including nasal prongs, and delivering the aerosolized active agent to the patient;
removing at least some of the deposits associated with the aerosolized active agent from the fluid flow connector;
re-aerosolizing the deposits to produce an additional volume of the aerosolized active agent; and
communicating the additional volume of the aerosolized active agent to the fluid flow connector for delivery to the same patient.

17. The method of claim 16, wherein the fluid flow connector comprises a trap for collecting deposits.

18. The method of claim 16, wherein the fluid flow connector comprises a port for retrieving deposits collected therein.

19. The method of claim 16, wherein the steps of removing at least some of the deposits associated with the first volume of the aerosolized active agent from the fluid flow connector and communicating a second volume of the aerosolized active agent to the fluid flow connector for delivery to the same patient are conducted substantially simultaneously.

20. The method of claim 16, wherein step of removing at least some of the deposits associated with the first volume of the aerosolized active agent from the fluid flow connector is conducted automatically via a collection reservoir connected to the fluid flow connector.

21. A method for delivering an aerosolized active agent to a patient, comprising the steps of:
obtaining the active agent as a mixture in a medium;
generating a stream of particles of the mixture with an aerosol generator to produce the aerosolized active agent;
collecting deposits separated from the aerosolized active agent;
delivering the aerosolized active agent to the patient; and
delivering at least some of the collected deposits to the patient.

22. A method for delivering an aerosolized active agent to a patient, comprising the steps of:
obtaining the active agent as a mixture in a medium;
generating a stream of particles of the mixture with an aerosol generator to produce the aerosolized active agent;
impacting the aerosolized active agent with a stream of gas in a substantially radially symmetric manner; and
delivering the stream of particles to the patient.

23. The method of claim 22, wherein the stream of gas has an initial temperature of about 37° Celsius to about 45° Celsius.

24. A method for delivering an aerosolized active agent to a patient, the method comprising the steps of:
obtaining the active agent as a mixture in a medium;
generating a stream of particles of the mixture with an aerosol generator to produce a first aerosol containing the active agent and the medium;
altering the characteristics of at least a portion of the first aerosol to produce a second aerosol; and
delivering the second aerosol to the patient.

25. The method of claim 24, wherein the step of altering the characteristics of at least a portion of the first aerosol to produce a second aerosol is accomplished at least in part by contacting the first aerosol with a controlled flow of gas.

26. The method of claim 24, wherein the mass median aerodynamic diameter of particles associated with the second aerosol is smaller than that of the particles associated with the first aerosol.

27. The method of claim 24, wherein the ratio of active agent to medium is greater in the second aerosol as compared to that in the first aerosol.

28. The method of claim 24, wherein the directional coherence of the stream of particles defining the second aerosol is greater than that defining the first aerosol.

29. A method of treating respiratory dysfunction in a patient comprising administering an aerosolized lung surfactant to the patient wherein the amount of surfactant deposited within the lung environment of the patient is effective to treat respiratory dysfunction in the patient.

30. The method of claim 29, wherein the patient is an infant.

31. A system useful for delivering an aerosolized active agent to a patient, the system comprising:
an aerosol generator for forming the aerosolized active agent;
a delivery means for delivering the aerosolized active agent; and
a trap interposed between the aerosol generator and delivery means for collecting deposits separated from the aerosolized active agent, wherein at least a portion of the trap is positioned substantially outside a main flow path of the aerosolized active agent.

32. The system of claim 31, wherein the trap is defined within a fluid flow connector and the delivery means are nasal prongs extending from the fluid flow connector.

33. The system of claim 31, further comprising a second trap spaced apart from the trap.

34. The system of claim 33, wherein the trap is defined within a fluid flow connector, and the second trap is defined within an aerosol conditioning vessel that is in fluid communication with the fluid flow connector.

35. A fluid flow connector useful for delivery of an aerosolized active agent to a patient, the connector comprising:
a chamber including an aerosol inlet, a delivery outlet, an aerosol flow path defined between the aerosol inlet and the delivery outlet, and an area for collecting deposits associated with the aerosolized active agent, the area for collecting deposits being located at least partially outside of the aerosol flow path so that deposits can be collected and substantially isolated from aerosolized active agent flowing through the fluid flow connector.

36. A fluid flow connector useful for the delivery of an aerosolized active agent to a patient, the connector comprising:
a chamber including an aerosol inlet, a delivery outlet, an aerosol flow path defined between the aerosol inlet and the delivery outlet, and a means for keeping deposits associated with the aerosolized active agent separated from the aerosol flow path.

37. The connector of claim 36, wherein the means for keeping deposits separated from the aerosol flow includes a concavity defined in a bottom portion of the chamber.

38. The connector of claim 36, wherein the means for keeping deposits separated from the aerosol flow includes a lip disposed proximate the delivery outlet.

39. A fluid flow connector useful for delivery of an aerosolized active agent to a patient, the connector comprising:
a chamber, an aerosol inlet for communicating the aerosolized active agent into the chamber, a delivery outlet for communicating the aerosolized active agent out of the chamber, and an aerosol flow path extending from the aerosol inlet to the delivery outlet, wherein the aerosolized active agent flows through the flow path at an angle that is less than about 90°, the angle of the flow path measured from a central axis point of the aerosol inlet where the aerosol inlet meets the chamber to a central axis point of the delivery outlet where the delivery outlet meets the chamber.

40. The connector of claim 39, wherein the angle of the flow path is less than about 75°.

41. The connector of claim 39, wherein the angle of the flow path is less than about 60°.

42. A fluid flow connector useful for delivery of an aerosolized active agent to a patient, the connector comprising:
a chamber including an aerosol inlet, a delivery outlet, and an internal surface on which deposits associated with the aerosolized active agent may impact, the internal surface being configured for either trapping the deposits and/or facilitating the communication of the deposits to the delivery outlet.

43. The connector of claim 42, wherein the internal surface includes a concave portion capable of trapping the deposits.

44. The connector of claim 42, wherein the internal surface is downwardly angled in a direction to the delivery outlet, so that gravity and/or surface characteristics are capable of communicating the deposits from an impact position to the delivery outlet.

45. The connector of claim 42, wherein the chamber further includes a ventilation gas inlet and a ventilation gas outlet.

46. The connector of claim 45, wherein a first fluid pathway extends between the aerosol inlet and the delivery outlet, and wherein the connector further comprises a baffle disposed between the ventilation gas inlet and the aerosol inlet to define a second fluid pathway for communicating ventilation gas to the delivery outlet and to delay intermixing of the ventilation gas with the aerosolized active agent flowing along the first fluid pathway.

47. The connector of claim 42, further comprising an aerosol conditioning vessel connected to the chamber, the aerosol conditioning vessel including a vessel inlet for receiving the aerosolized active agent from an aerosol generator, and a vessel outlet in fluid communication with the chamber aerosol inlet.

48. The connector of claim 47, wherein the aerosol conditioning vessel is permanently connected to the chamber.

49. The connector of claim 47, wherein a portion of the chamber and a portion of the aerosol conditioning vessel are integrally formed.

50. The connector of claim 47, wherein the aerosol conditioning vessel includes a plurality of gas inlets that are radially symmetrically disposed about the aerosol conditioning vessel.

51. The connector of claim 47, wherein the aerosol conditioning vessel includes a trap for accepting deposits associated with the aerosolized active agent.

52. The connector of claim 42, further comprising an active agent concentrating chamber in fluid communication with the aerosol inlet.

53. The connector of claim 52, wherein a one-way valve is disposed between the active agent concentrating chamber and the aerosol inlet.

54. The connector of claim 42, wherein the chamber includes one or more baffles for directing fluid flow therein.

55. The connector of claim 42, further comprising a collection reservoir disposed below and in fluid communication with the chamber for accepting deposits associated with the aerosolized active agent.

56. A fluid flow connector useful for delivery of an aerosolized active agent to a patient, the connector comprising:
a chamber including an aerosol inlet, a delivery outlet, a ventilation gas inlet and a ventilation gas outlet, wherein the aerosol inlet and the delivery outlet are substantially parallel to each other.

57. The connector of claim 56, wherein the aerosol inlet is laterally offset from the delivery outlet.

58. A system for delivering an aerosolized active agent to a patient, the system comprising:
an aerosol generator;
a fluid flow connector connected to the aerosol generator, the fluid flow connector including chamber, an aerosol inlet, a delivery outlet, and a trap for collecting deposits associated with the aerosolized active agent, wherein an aerosol flow path is defined between the aerosol inlet and the delivery outlet and wherein the aerosol flow path is devoid of angles greater than or equal to about 90°.

59. The system of claim 58 further comprising:
a pair of nasal prongs connected to the delivery outlet, each of the nasal prongs having an internal diameter that is less than or equal to about 10 mm.

60. The system of claim 59 wherein the each of the nasal prongs have an internal diameter that is less than or equal to about 5 mm.

61. The system of claim 59, wherein the each of the nasal prongs have an internal diameter that is less than or equal to about 3 mm.
